(19) United States
(12) Patent Application Publication
(10) Pub. No.: US 2004/0185506 A1
(43) Pub. Date: Sep. 23, 2004

(54) Epitope mapping using nuclear magnetic resonance

(76) Inventor: George A. Heavner, Malvern, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/393,926

(22) Filed: Mar. 21, 2003

(51) Int. Cl. G01N 33/53; G01N 33/567

(52) U.S. Cl. 435/7.2

(57) ABSTRACT

This invention relates to methods for mapping or otherwise identifying the amino acid sequence and conformation of a portion of a protein that is involved in ligand binding. This invention finds utility in the process of elucidating the amino acid sequence and conformation of an epitope of, for example, an antigen or an antibody that binds to the antigen.

### FIELD OF INVENTION

This invention relates to methods for mapping or otherwise identifying the amino acid sequence and conformation of a potion of a protein involved in ligand binding.

### BACKGROUND OF THE INVENTION

When two biological molecules bind to each other, such as when an antibody binds to an antigen or a ligand binds to a receptor, there are discrete interactions between binding domains of the biological molecules themselves. For example, regarding antibody/antigen binding, there are interactions between the amino acids residing primarily within the complementarity determining regions of an antibody and, in the case where the antigen is a protein, the amino acids in the epitope of the antigen.

Several techniques have been used to determine the identity, conformation, or amino acid make-up of various binding domains. These include the synthesis of peptides on pins or in spots on membrane, the use of synthetic peptides or proteolytic fragments of the antigen in ELISA or competition assays, analysis of the binding of the antibody to antigens in which point mutations have been made, phage display libraries of random peptide or antigen fragments sequences, proteolysis of antigens in the presence or absence of antibody with mass spectral analysis of either binding or non-binding fragments, and X-ray crystallography. Many of these methods are expensive and time consuming, requiring many steps before meaningful results are available. The invention herein provides a new technique for the identification of amino acid sequences involved in molecular interaction between biological molecules using nuclear magnetic resonance (NMR).

### SUMMARY OF THE INVENTION

The invention herein provides new methods of characterizing a binding domain of a labeled, recombinant protein comprising comparing a nuclear magnetic resonance spectrum of a labeled, recombinant protein in the absence of a ligand with a nuclear magnetic resonance spectrum of a labeled, recombinant protein in the presence of a ligand, wherein the binding domain is characterized after the comparing step is undertaken one or more times.

In one embodiment of the present invention, the recombinant protein can be recombinant protein labeled at multiple sites or a recombinant protein labeled at alternate sites.

In another embodiment of the present invention, the recombinant protein can be tissue plasminogen activator receptor and the ligand can be tissue plasminogen activator.

In another embodiment of the present invention, the recombinant protein can be tumor necrosis factor and the ligand can be any antibody that binds to tumor necrosis factor.

In another embodiment of the present invention, the recombinant protein can be erythropoietin receptor and the ligand can be erythropoietin or an erythropoietin mimetic.

In another embodiment of the present invention, the recombinant protein or the ligand can be a diagnostic compound or a therapeutic compound. The diagnostic or therapeutic compound can be any of the immunoglobulins, fragments of immunoglobulins, integrins, antigens, growth factors, cell cycle proteins, cytokines, hormones, neurotransmitters, receptors or fusion proteins thereof, blood proteins, antimicrobials, chemokines, or any structural or functional analog thereof.

In another embodiment of the present invention, the immunoglobulin can be IgG1, IgG2, IgG3, IgG4, IgΛ1, IgΛ2, slgΛ, IgD, IgE, or any structural or functional analog thereof.

In another embodiment of the present invention, the fragment of immunoglobulin can be F(ab′)₂, Fab′, Fab, Fc, Facb, pFc′, Fd, FV, or any structural or functional analog thereof.

In another embodiment of the present invention, the integrin can be αv, β1, β2, β3, β4, β5, or any structural or functional analog thereof.

In another embodiment of the present invention, the antigen can be any of the cancer-specific antigens, viral antigens, bacterial antigens, chemical antigens, or any structural or functional analog thereof.

In another embodiment of the present invention, the growth factor can be any of the insulin-like growth factors, platelet-derived growth factors, fibroblast growth factor 2, epidermal growth factors, tumor growth factors, nerve growth factors, vascular endothelial growth factors, or any structural or functional analog thereof.

In another embodiment of the present invention, the cell cycle protein can be p13, p27, p34, p53, p60, p80, histone H1, any of the centrosomal proteins, any of the lamins, any of the CDKs, any of the CDK inhibitors, any of the tumor suppressor genes, or any structural or functional analog thereof.

In another embodiment of the present invention, the cytokine can be IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, any of the interferons, any of the interleukins, any of the lymphokines, any of the cell signal molecules, or any structural or functional analog thereof.

In another embodiment of the present invention, the hormone can be any of the parathyroid hormones, follicle stimulating hormones, estrogens, progesterones, testosterones, aldosterones, antidiuretic hormones, cortisols, human chorionic gonadotropins, parathormones, growth hormones, secretins, thyroid hormones, or any structural or functional analog thereof.

In another embodiment of the present invention, the neurotransmitter can be GABA, any of the enkephalins, or any structural or functional analog thereof.

In another embodiment of the present invention, the receptor or fusion protein thereof can be any of the chemokine receptors, cytokine receptors, cell cycle protein receptors, growth factor receptors, hormone receptors, neurotransmitter receptors, fusion proteins to any one of the foregoing, or any structural or functional analog thereof.

In another embodiment of the present invention, the blood protein can be any of the TPOs, any of the tPAs, or any structural or functional analog thereof.

In another embodiment of the present invention, the antimicrobial can be any of the antibiotics, antifungals, antiprotozoals, antivirals, or any structural or functional analog thereof.

In another embodiment of the present invention, the labeled, recombinant protein can be an isotopically labeled protein. The label can be ¹⁵N, ¹³C, ³H, ³³S, ¹⁷O, or ²H.

In another embodiment of the present invention, the comparing step can be accomplished via a computer. The comparing step may also involve data obtained from X-ray crystallography analysis.

The invention herein also provides new methods of characterizing a binding domain of a ligand comprising comparing a nuclear magnetic resonance spectrum of a labeled, recombinant ligand of a ligand-binding compound in the absence of a ligand-binding compound with a nuclear magnetic resonance spectrum of a labeled, recombinant ligand of a ligand-binding compound in the presence of a ligand-binding compound, wherein the binding domain can be characterized after the comparing step is undertaken one or more times.

In one embodiment of the present invention, the labeled, recombinant ligand can be a labeled, recombinant ligand labeled at multiple sites or a labeled, recombinant ligand labeled at alternate sites.

In another embodiment of the present invention, the ligand-binding compound can be tissue plasminogen activator receptor and the labeled, recombinant ligand can be tissue plasminogen activator.

In another embodiment of the present invention, the ligand-binding compound can be tumor necrosis factor and the labeled, recombinant ligand can be any antibody that binds to tumor necrosis factor.

In another embodiment of the present invention, the ligand-binding compound can be erythropoietin receptor and the labeled, recombinant ligand can be erythropoietin or an erythropoietin mimetic.

In another embodiment of the present invention, the ligand-binding compound or the labeled, recombinant ligand can be a diagnostic compound or a therapeutic compound. The diagnostic or therapeutic compound can be any of the immunoglobulins, fragments of immunoglobulins, integrins, antigens, growth factors, cell cycle proteins, cytokines, hormones, neurotransmitters, receptors or fusion proteins thereof, blood proteins, antimicrobials, chemokines, or any structural or functional analog thereof.

In another embodiment of the present invention, the immunoglobulin can be IgG1, IgG2, IgG3, IgG4, IgA1, IgA2, slgA, IgD, IgE, or any structural or functional analog thereof.

In another embodiment of the present invention, the fragment of immunoglobulin can be F(ab′)₂, Fab′, Fab, Fc, Facb, pFc′, Fd, FV, or any structural or functional analog thereof.

In another embodiment of the present invention, the integrin can be αv, β1, β2, β3, β4, β5, or any structural or functional analog thereof.

In another embodiment of the present invention, the antigen can be any of the cancer-specific antigens, viral antigens, bacterial antigens, chemical antigens, or any structural or functional analog thereof.

In another embodiment of the present invention, the growth factor can be any of the insulin-like growth factors, platelet-derived growth factors, fibroblast growth factor 2, epidermal growth factors, tumor growth factors, nerve growth factors, vascular endothelial growth factors, or any structural or functional analog thereof.

In another embodiment of the present invention, the cell cycle protein can be p13, p27, p34, p53, p60, p80, histone H1, any of the centrosomal proteins, any of the lamins, any of the CDKs, any of the CDK inhibitors, any of the tumor suppressor genes, or any structural or functional analog thereof.

In another embodiment of the present invention, the cytokine can be IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, any of the interferons, any of the interleukins, any of the lymphokines, any of the cell signal molecules, or any structural or functional analog thereof.

In another embodiment of the present invention, the hormone can be any of the parathyroid hormones, follicle stimulating hormones, estrogens, progesterones, testosterones, aldosterones, antidiuretic hormones, cortisols, human chorionic gonadotropins, parathormones, growth hormones, secreting, thyroid hormones, or any structural or functional analog thereof.

In another embodiment of the present invention, the neurotransmitter can be GABA, any of the enkephalins, or any structural or functional analog thereof.

In another embodiment of the present invention, the receptor or fusion protein thereof can be any of the chemokine receptors, cytokine receptors, cell cycle protein receptors, growth factor receptors, hormone receptors, neurotransmitter receptors, fusion proteins to any one of the foregoing, or any structural or functional analog thereof.

In another embodiment of the present invention, the blood protein can be any of the TPOs, any of the tPAs, or any structural or functional analog thereof.

In another embodiment of the present invention, the antimicrobial can be any of the antibiotics, antifungals, antiprotozoals, antivirals, or any structural or functional analog thereof.

In another embodiment of the present invention, the ligand-binding compound can be an isotopically labeled protein. The label can be ¹⁵N, ¹³C, ³H, ³³S, ¹⁷O, or ²H.

In another embodiment of the present invention, the comparing step can be accomplished via a computer. The comparing step may also involve data obtained from X-ray crystallography analysis.

Further, the invention herein provides new methods of characterizing a binding domain of a labeled ligand-binding compound comprising comparing a nuclear magnetic resonance spectrum of a labeled, ligand-binding compound in the absence of a ligand with a nuclear magnetic resonance spectrum of a labeled, ligand-binding compound in the presence of a ligand, wherein the binding domain can be characterized after the comparing step is undertaken one or more times.

In one embodiment of the present invention, the ligand-binding compound can be a ligand-binding compound labeled at multiple sites or a ligand-binding compound labeled at alternate sites.

In another embodiment of the present invention, the ligand-binding compound can be tissue plasminogen activator receptor and the ligand can be tissue plasminogen activator.

In another embodiment of the present invention, the ligand-binding compound can be tumor necrosis factor and the ligand can be any antibody that binds to tumor necrosis factor.

In another embodiment of the present invention, the ligand-binding compound can be erythropoietin receptor and the ligand can be erythropoietin or an erythropoietin mimetic.

In another embodiment of the present invention, the ligand-binding compound or the ligand can be a diagnostic compound or a therapeutic compound. The diagnostic or therapeutic compound can be any of the immunoglobulins, fragments of immunoglobulins, integrins, antigens, growth factors, cell cycle proteins, cytokines, hormones, neurotransmitters, receptors or fusion proteins thereof, blood proteins, antimicrobials, chemokines, or any structural or functional analog thereof.

In another embodiment of the present invention, the immunoglobulin can be IgG1, IgG2, IgG3, IgG4, IgA1, IgA2, slgA, IgD, IgE, or any structural or functional analog thereof.

In another embodiment of the present invention, the fragment of immunoglobulin can be F(ab′)₂, Fab′, Fab, Fc, Facb, pFc′, Fd, FV, or any structural or functional analog thereof.

In another embodiment of the present invention, the integrin can be αv, β1, β2, β3, β4, β5, or any structural or functional analog thereof.

In another embodiment of the present invention, the antigen can be any of the cancer-specific antigens, viral antigens, bacterial antigens, chemical antigens, or any structural or functional analog thereof.

In another embodiment of the present invention, the growth factor can be any of the insulin-like growth factors, platelet-derived growth factors, fibroblast growth factor 2, epidermal growth factors, tumor growth factors, nerve growth factors, vascular endothelial growth factors, or any structural or functional analog thereof.

In another embodiment of the present invention, the cell cycle protein can be p13, p27, p34, p53, p60, p80, histone H1, any of the centrosomal proteins, any of the lamins, any of the CDKs, any of the CDK inhibitors, any of the tumor suppressor genes, or any structural or functional analog thereof.

In another embodiment of the present invention, the cytokine can be IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, any of the interferons, any of the interleukins, any of the lymphokines, any of the cell signal molecules, or any structural or functional analog thereof.

In another embodiment of the present invention, the hormone can be any of the parathyroid hormones, follicle stimulating hormones, estrogens, progesterones, testosterones, aldosterones, antidiuretic hormones, cortisols, human chorionic gonadotropins, parathormones, growth hormones, secreting, thyroid hormones, or any structural or functional analog thereof.

In another embodiment of the present invention, the neurotransmitter can be GABA, any of the enkephalins, or any structural or functional analog thereof.

In another embodiment of the present invention, the receptor or fusion protein thereof can be any of the chemokine receptors, cytokine receptors, cell cycle protein receptors, growth factor receptors, hormone receptors, neurotransmitter receptors, fusion proteins to any one of the foregoing, or any structural or functional analog thereof.

In another embodiment of the present invention, the blood protein can be any of the TPOs, any of the tPAs, or any structural or functional analog thereof.

In another embodiment of the present invention, the antimicrobial can be any of the antibiotics, antifungals, antiprotozoals, antivirals, or any structural or functional analog thereof.

In another embodiment of the present invention, the labeled, ligand-binding compound can be an isotopically labeled compound. The label can be ¹⁵N, ¹³C, ³H, ³³S, ¹⁷O, or ²H.

In another embodiment of the present invention, the comparing step can be accomplished via a computer. The comparing step may also involve data obtained from X-ray crystallography analysis.

The invention herein also provides any of the binding domains characterized by any of the methodologies of the present invention.

The invention herein also provides databases comprising any and/or all of the data collected via any of the methodologies of the present invention. Such databases may or may not be managed by a computer system. Such databases may or may not appear within recordable media.

Also provided by the present invention are business methods comprising performing for a customer any of the methodologies of the present invention.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that can become apparent to those skilled in the art from this detailed description.

### DETAILED DESCRIPTION OF THE INVENTION

It is to be understood that this invention is not limited to the particular methodology, protocols, constructs, formulae and reagents described and as such may vary. It is also to be understood that the terminology used herein is for the purpose of describing particular embodiments only, and is not intended to limit the scope of the present invention.

It must be noted that as used herein and in the appended claims, the singular forms “a,” “and,” and “the” include plural reference unless the context clearly dictates otherwise. Thus, for example, reference to “a protein” is a reference to one or more proteins and includes equivalents thereof known to those skilled in the art, and so forth. Further, reference to a “ligand-binding compound” is a reference to any substance, including but not limited to any recombinant protein, that binds a ligand.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

This invention provides a new procedure for the identification of protein binding domains using recombinant proteins or ligands enriched with isotopically labeled amino acids, in which precise identification of NMR signals is not always required. These binding domains, as the term is used herein, can be the binding regions of any biological molecules, including antibodies, antigens, ligands, and receptors. For convenience, the term ‘epitope’ is used synonymously with binding domain in many instances throughout the present specification.

NMR is a technique that identifies specific atoms (generally ¹H, ¹³C, and ¹⁵N), and hence identifies amino acid residues, based on the local environment of these atoms within the amino acid molecule. Additionally, carbon and nitrogen NMR spectra are less complex than proton spectra, but natural abundance of the required nuclei can limit sensitivity. With large proteins, there can be considerable overlap in the spectra between atoms in similar environments. Complete assignment of most or all resonances can be done for large proteins, however, given sufficient time and instruments of high enough resolution.

When a biological molecule binds to a protein, the local environment of some amino acids changes. Those amino acids affected most profoundly are those most likely involved with ligand/protein contact, i.e., the binding domain. Theoretically, it is possible to identify the amino acids of the binding domain by making the NMR assignments for both the protein and the ligand in bound and unbound states, then determining with amino acids have shifted. The complexity of the NMR spectra of an ligand/protein complex makes such an analysis extremely difficult if not impossible, however, with current instruments and methods and is not, therefore, applicable to routine epitope identification. See, e.g., REES ET AL., in PROTEIN & PHARM. ENG'G 35-53 (Craik et al. eds., 1990).

The present invention provides a simple method for using NMR to determine the amino acids involved in a binding domain. More specifically, using recombinant technology, a ligand, ligand-binding compound, or a recombinant protein, such as an antibody, antigen or receptor, is expressed in a medium in which a single amino acid has been replaced with an isotopically labeled (for example, ¹⁵N, ¹³C, ³H, ³³S, ¹⁷O, or ²H) counterpart. The ligand, ligand-binding compound, or recombinant protein generated in this manner will have the identical structure and functionality as its unlabeled counterpart.

The ¹⁵N, ¹³C, ³H, ³³S, ¹⁷O, or ²H NMR spectra are then run on the ligand, ligand-binding compound, or recombinant protein, separately in the presence and absence of the ligand's binding partner or the recombinant protein's or ligand-binding compound's ligand. The low natural abundance of the resonating nuclei of the unlabeled amino acids provides 5 simplified spectra such that a decoupled spectra will exhibit singlets of ¹⁵N spectra and singlets or simple patterns for ¹³C, ³H, ³³S, ¹⁷, or ²H spectra, depending on whether the amino acids were uniformly or specifically labeled. In those instances where the labeled amino acid is involved in binding with the target, a shift in resonance is seen.

As an example, if a protein of 200 amino acids contained ten ¹⁵N-labeled alanines, then ten singlets would be seen in the ¹⁵N spectra. If, when bound to the ligand, two of these spectra are shifted, this reflects the alteration in the residues' local environment. From this, their location in the binding domain can be inferred. The specific location of the two alanines in the sequence, for example, might not be determined from this single spectrum, but when this process is repeated twenty times with a different amino acid labeled each time, the, composition of the binding domain is identified. Moreover, because the sequence of the protein is known (in a preferred embodiment the protein is produced recombinantly), the location and specific sequence of the binding domain is determined by comparing the known amino acid sequence with the amino acids identified by the NMR studies.

In another embodiment of the invention, data obtained from the methodologies involving NMR can be considered in light of data obtained from X-ray crystallography analyses. For example, conclusions reached via the NMR-related methodologies of the present invention can be confirmed or partially supported by conclusions reached via X-ray crystallography analyses. Hence, research methodologies comprising NMR-related methodologies and X-ray crystallography analyses are fully within the scope of the present invention. Additionally, in another embodiment of the invention, molecular modeling or algorithms that predict surface-exposed sequences on proteins may provide useful information for epitope identification.

The methodologies taught herein offer several advantages over current procedures for epitope identification.

For example, current methods using synthetic peptides (including, but not limited to, pin, spot, or solution and ELISA, or competition assays) or phage or mere fragments of antigen can miss conformational epitopes. Current variations of the spot synthesis (e.g., matrix of peptides) are purportedly better in identifying conformational epitopes, but the number of peptides required for this analysis increases exponentially with the number of amino acids in the protein to the point that approximately two million peptides would be required to characterize a protein of molecular weight 40 kD. See WO 00/72880 A2; Kleinjung et al., 53 BIOPOLYMERS 113-28 (2000); Tugarinov et al., 8 STRUCTURE 385-95 (2000); WINKLER ET AL., in PEPTIDES 1996, 913-14 (Robert Ramage and Roger Epton eds., 1998); Tsang et al., 10 J. MOL. RECOG. 256-62 (1997); Kustanovich & Zvi, 66 EPITOPE MAPPING PROTOCOLS 25-37 (1996); Hubbard et al., 6 PROTEIN SCI. 1945-52 (1997); Zvi et al., 229 EUR. J. BIOCHEM. 178-87 (1995); Cung et al., 31 BIOPOLYMERS 769-76 (1991); Bechtel et al., 11 BULL. MAGN. RESON. 421 (1989); Huang et al., 281 J. MOL. BIOL. 61-67 (1998); Zvi & Anglister, 5 LETTERS IN PEPTIDE SCI. 357-64 (1998). The NMR procedure of the present invention, because it uses the intact protein, will detect conformational epitopes as readily as linear epitopes.

Another current approach, proteolysis, in combination with mass spectrometry, can identify some conformational epitopes, but the technique destroys the protein and increasingly larger amounts of protein are needed as the molecular weight increases. Bundle et al., 33 BIOCHEM. 5183-92 (1994). In comparison, the NMR procedure described herein is non-destructive. Indeed, if the labeled protein is in short supply it can be recovered after each experiment and reused to map, for example, another ligand.

The instant invention is also advantageous over point mutations or ‘alanine scans’ of proteins, which have been used for the identification of both linear and conformational epitope mapping. Cung et al., 11 BULL. MAGN. RESON. 381 (1989). The difficulty of this approach is that each protein requires its own DNA for expression, and for each mutation it must be determined if the target protein has refolded properly upon purification. In contrast, the NMR procedure of the present invention uses the same DNA for all labeled protein, and the labeled and unlabeled proteins are purified and fold identically.

It has been suggested that X-ray crystallography can be used to identify the binding sites of organic solvents on macromolecules. WO 99/54504. Its drawbacks include the extensive amount of time involved, the amount of protein required, the difficulty of growing a diffraction grade crystal, and the fact that each antibody to the same antigen requires a new protein and a new crystal. Moreover, this approach is not a screening method for rapidly testing many compounds that are chemically diverse, but is limited to mapping the binding sites of only a few organic molecules due to the long time needed to determine the individual crystal structures.

The nature and source of the compounds involved within the methodologies of the present invention are not limited. For example, the NMR techniques of the present invention can be used to determine the binding domains of an immunoglobulin or fragment thereof which binds an antigen, a cytokine, an integrin, a growth factor, a cell cycle protein, a hormone, a neurotransmitter, a receptor or fusion protein thereof, a blood protein, an antimicrobial, any fragment thereof, and any structural or functional analog thereof. The following is a general discussion of the variety of proteins, peptides and biological molecules that can be used in the in accordance with the teachings herein. These descriptions do not serve to limit the scope of the invention, but rather illustrate the breadth of the invention.

One embodiment of the methodologies of the present invention involves human or non-human monoclonal antibodies or antibody fragments. Specifically, these antibodies (immunoglobulins) can be isolated, recombinant and/or synthetic human, primate, rodent, mammalian, chimeric, humanized or CDR-grafted, antibodies and anti-idiotype antibodies thereto. Such antibodies or antibody fragments can be produced by enzymatic cleavage, synthetic techniques, or recombinant techniques as known in the art and/or as described herein. Additionally, these antibodies can also be produced in a variety of truncated forms in which various portions of antibodies are joined together chemically by conventional techniques, or prepared as a contiguous protein using genetic engineering techniques. As used presently, an “antibody,” “antibody fragment,” “antibody variant,” “Fab,” and the like, include any protein- or peptide-containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof, or at least one portion of a receptor or binding protein, which can be identified using the techniques of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one target activity or binding, or with receptor activity or binding, in vitro, in situ and/or in vivo.

Another embodiment of methodologies of the present invention may involve antibodies, or functional equivalents thereof, that are “human,” such that they are substantially non-immunogenic in humans. These antibodies can be prepared through any of the methodologies described herein, including the use of transgenic animals, genetically engineered to express human antibody genes. For example, immunized transgenic mice (xenomice) that express either fully human antibodies, or human variable regions have been described. WO 96/34096. In the case of xenomice, the antibodies produced include fully human antibodies and can be obtained from the animal directly (e.g., from serum), or from immortalized B-cells derived from the animal, or from the genes encoding the immunoglobulins with human variable regions can be recovered and expressed to obtain the antibodies directly or modified to obtain analogs of antibodies such as, for example, Fab or single chain Fv molecules. Id. See also, e.g., U.S. patent application Ser. No. 09/920,137; U.S. Provisional Patent Application Nos. 60/223,369; 60/236,826.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. The methodologies of the present invention thus encompass the use of antibody fragments capable of binding to a biological molecule (such as an antigen or receptor) or portions thereof, including but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments. See, e.g., CURRENT PROTOCOLS IN IMMUNOLOGY (Colligan et al., eds., John Wiley & Sons, Inc., NY, 1994-2001).

As with antibodies, other peptide moieties that bind a particular target protein or other biological molecule (target-binding peptides) are utilized within the methodologies disclosed herein. Such target-binding peptides can be isolated from tissues and purified to homogeneity, or isolated from cells which contain the target-binding protein, and purified to homogeneity. Once isolated and purified, such target-binding peptides can be sequenced by well-known methods. From these amino acid sequences, DNA probes can be produced and used to obtain mRNA, from which cDNA can be made and cloned by known methods. Other well-known methods for producing cDNA are known in the art and may effectively be used. In general, any target-binding peptide can be isolated from any, cell or tissue expressing such proteins using a cDNA probe such as the probe described above, isolating mRNA and transcribing the mRNA into cDNA. Thereafter, the protein can be produced by inserting the cDNA into an expression vector, such as a virus, plasmid, cosmid, or other vector, inserting the expression vector into a cell, proliferating the resulting cells, and isolating the expressed target-binding protein from the medium or from cell extract as described above. Alternatively, target-binding peptides can be chemically synthesized using the sequence described above and an amino acid synthesizer, or manual synthesis using chemical conditions well known to form peptide bonds between selected amino acids. Analogues and fragments of target-binding proteins can be produced by chemically modification or by genetic engineering. These fragments and analogues may then be tested for target-binding activity using known methods. See, e.g., U.S. Pat. No. 5,808,029 to Brockhaus et al., issued Sep. 15, 1998.

Alternatively, target-binding peptides, including antibodies, can be identified using various library screening techniques. For example, peptide library screening takes advantage of the fact that molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”). Phage display peptide libraries have emerged as a powerful method in identifying such peptide agonists and antagonists. In such libraries, random peptide sequences are displayed by fusion with coat proteins of filamentous phage. Typically, the displayed peptides are affinity-eluted against an immobilized extracellular domain of an antigen or receptor. The retained phages can be enriched by successive rounds of affinity purification and repropagation. The best binding peptides can be sequenced to identify key residues within one or more structurally related families of peptides. The peptide sequences may also suggest which residues can be safely replaced by alanine scanning or by mutagenesis at the DNA level. Mutagenesis libraries can be created and screened to further optimize the sequence of the best binders. See, e.g., WO 0024782, published May 4, 2000, and the references cited therein; U.S. Pat. No. 6,090,382 to Salfeld et al., issued Jul. 18, 2000; WO 93/06213, to Hoogenboom et al., published Apr. 1, 1993.

Other display library screening methods are known as well. For example, E. coli displays employ a peptide library fused to either the carboxyl terminus of the lac-repressor or the peptidoglycan-associated lipoprotein, and expressed in E. coli. Ribosome display involves halting the translation of random RNAs prior to ribosome release, resulting in a library of polypeptides with their associated RNAs still attached. RNA-peptide screening employs chemical linkage of peptides to RNA. Additionally, chemically derived peptide libraries have been developed in which peptides are immobilized on stable, non-biological materials, such as polyethylene rods or solvent-permeable resins. Another chemically derived peptide library uses photolithography to scan peptides immobilized on glass slides. These methods of chemical-peptide screening can be advantageous because they allow use of D-amino acids and other unnatural analogues, as well as non-peptide elements. See WO 0024782, published May 4, 2000, and the references cited therein.

Moreover, structural analysis of binding domains may also be used to suggest peptides that mimic the binding activity of large protein ligands. In such an analysis, the crystal structure may suggest the identity and relative orientation of critical residues of the large protein ligand, from which a peptide can be designed. These analytical methods may also be used to investigate the interaction between, for example, a receptor protein and peptides selected by phage display, which may suggest further modification of the peptides to increase binding affinity. Thus, conceptually, one may discover peptide mimetics of any protein using phage display and the other methods mentioned above. For example, these methods provide for epitope mapping, for identification of critical amino acids in protein-protein interactions, and as leads for the discovery of new therapeutic agents. See WO 00/24782, published May 4, 2000, and the references cited therein.

The methods of the instant invention may also be used to characterize the binding of synthetic peptides to target receptors or antigens. For example, solution-phase synthesis has been used to create the eptifibatide molecule that binds the platelet receptor glycoprotein IIb/IIIa of human platelets, thus inhibiting platelet aggregation. Eptifibatide, sold commercially as INTEGRILIN® (COR Therapeutics, Belmont, Calif.), is a cyclic heptapeptide containing six amino acids and one mercaptopropionyl (des-amino cysteinyl) residue.

In one embodiment of the present invention, the methods described herein can be used to determine the binding domain of a growth factor to its receptor or other binding target. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following Table 1 presents several factors, but is not intended to be comprehensive or complete, yet introduces some of the more commonly known factors and their principal activities.

TABLE 1
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different protein |
| | | receptor has intrinsic tyrosine | chains, A and B, form |
| | | kinase activity. | 3 distinct dimer |
| | | | forms. |
| Epidermal | Submaxillary gland, | promotes proliferation of | EGF receptor has |
| Growth Factor | Brunners gland. | mesenchymal, glial and | tyrosine kinase |
| (EGF) | | epithelial cells | activity, activated in |
| | | | response to EGF |
| | | | binding. |
| Fibroblast | Wide range of cells; | Promotes proliferation of | Four distinct |
| Growth Factor | protein is associated with | many cells including skeletal | receptors, all with |
| (FGF) | the ECM; nineteen family | and nervous system; inhibits | tyrosine kinase |
| | members. Receptors | some stem cells; induces | activity. FGF |
| | widely distributed in | mesodermal differentiation. | implicated in mouse |
| | bone, implicated in | Non-proliferative effects | mammary tumors and |
| | several bone-related | include regulation of pituitary | Kaposi's sarcoma. |
| | diseases. | and ovarian cell function. | |
| NGF | | Promotes neurite outgrowth | Several related |
| | | and neural cell survival | proteins first |
| | | | identified as protooncogenes; |
| | | | trkA |
| | | | (trackA), trkB, trkC |
| Erythropoietin | Kidney | Promotes proliferation and | Also considered a |
| (Epo) | | differentiation of erythrocytes | ‘blood protein,’ and a |
| | | | colony stimulating |
| | | | factor. |
| Transforming | Common in transformed | Potent keratinocyte growth | Related to EGF. |
| Growth Factor a | cells, found in | factor. | |
| (TGF-α) | macrophages and | | |
| | keratinocytes | | |
| Transforming | Tumor cells, activated | Anti-inflammatory (suppresses | Large family of |
| Growth Factor v | TH₁ cells (T-helper) and | cytokine production and class | proteins including |
| (TGF-β) | natural killer (NK) cells | II MHC expression), | activin, inhibin and |
| | | proliferative effects on many | bone morpho-genetic |
| | | mesenchymal and epithelial | protein. Several |
| | | cell types, may inhibit | classes and |
| | | macrophage and lymphocyte | subclasses of cell- |
| | | proliferation. | surface receptors |
| Insulin-Like | Primarily liver, produced | Promotes proliferation of | Related to IGF-II and |
| Growth Factor-I | in response to GH and | many cell types, autocrine and | proinsulin, also called |
| (IGF-I) | then induces subsequent | paracrine activities in addition | Somatomedin C. |
| | cellular activities, | to the initially observed | IGF-I receptor, like |
| | particularly on bone | endocrine activities on bone. | the insulin receptor, |
| | growth | | has intrinsic tyrosine |
| | | | kinase activity. IGF-I |
| | | | can bind to the |
| | | | insulin receptor. |
| Insulin-Like | Expressed almost | Promotes proliferation of | IGF-II receptor is |
| Growth | exclusively in embryonic | many cell types primarily of | identical to the |
| Growth | and neonatal tissues. | fetal origin. Related to IGF-I | mannose-6-phosphate |
| Factor-II | | and proinsulin. | receptor that is |
| (IGF-II) | | | responsible for the |
| | | | integration of |
| | | | lysosomal enzymes |

Additional growth factors that can be utilized within the methodologies of the present invention include insulin and proinsulin (U.S. Pat. No. 4,431,740); Activin (Vale et al., 321 NATURE 776 (1986); Ling et al., 321 NATURE 779 (1986)); Inhibin (U.S. Pat. Nos. 4,740,587; 4,737,578); and Bone Morphongenic Proteins (BMPs) (U.S. Pat. No. 5,846,931; WOZNEY, CELLULAR & MOLECULAR BIOLOGY OF BONE 131-167 (1993)).

Additional growth factors that can be utilized within the methodologies of the present invention include Activin (Vale et al., 321 NATURE 776 (1986); Ling et al., 321 NATURE 779 (1986)), Inhibin (U.S. Pat. Nos. 4,737,578; 4,740,587), and Bone Morphongenic Proteins (BMPs) (U.S. Pat. No. 5,846,931; WOZNEY, CELLULAR & MOLECULAR BIOLOGY OF BONE 131-67 (1993)).

In another embodiment, the methodologies of the present invention can be used to characterize the binding domain of a cytokine or cytokine receptor. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), because they are not only secreted by leukocytes, but are also able to affect the cellular responses of leukocytes. More specifically, interleukins are growth factors targeted to cells of hematopoietic origin. The list of identified interleukins grows continuously. See, e.g., U.S. Pat. No. 6,174,995; U.S. Pat. No. 6,143,289; Sallusto et al., 18 ANNU. REV. IMMUNOL. 593 (2000); Kunkel et al., 59 J. LEUKOCYTE BIOL. 81 (1996).

Additional growth factor/cytokines encompassed in the methodologies of the present invention include pituitary hormones such as CEA, FSH, FSH α, FSH β, Human Chorionic Gonadotrophin (HCG), HCG α, HCG β, uFSH (urofollitropin), GH, LH, LH α, LH β, PRL, TSH, TSH α, TSH β, and CA, parathyroid hormones, follicle stimulating hormones, estrogens, progesterones, testosterones, or structural or functional analog thereof. All of these proteins and peptides are known in the art. Many can be obtained commercially from, e.g., Research Diagnostics, Inc. (Flanders, N.J.).

The cytokine family also includes tumor necrosis factors, colony stimulating factors, and interferons. See, e.g., Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984); R & D Systems, CYTOKINE MINI-REVIEWS, at http://www.rndsystems.com.

Several cytokines are introduced, briefly, in Table 2 below.

TABLE 2
Cytokines

| Cytokine | Principal Source | Primary Activity |
|---|---|---|
| Interleukins | Primarily macrophages but also | Costimulation of APCs and T cells; |
| IL1-α and -β | neutrophils, endothelial cells, smooth | stimulates IL-2 receptor production and |
| | muscle cells, glial cells, astrocytes, B- | expression of interferon-γ; may induce |
| | and T-cells, fibroblasts, and | proliferation in non-lymphoid cells. |
| | keratinocytes. | |
| IL-2 | CD4+ T-helper cells, activated TH₁ | Major interleukin responsible for clonal |
| | cells, NK cells. | T-cell proliferation. IL-2 also exerts |
| | | effects on B-cells, macrophages, and |
| | | natural killer (NK) cells. . IL-2 receptor |
| | | is not expressed on the surface of resting |
| | | T-cells, but expressed constitutively on |
| | | NK cells, that will secrete TNF-α, IFN-g |
| | | and GM-CSF in response to IL-2, which |
| | | in turn activate macrophages. |
| IL-3 | Primarily T-cells | Also known as multi-CSF, as it stimulates |
| | | stem cells to produce all forms of |
| | | hematopoietic cells. |
| IL-4 | TH₂ and mast cells | B cell proliferation, eosinophil and mast |
| | | cell growth and function, IgE and class II |
| | | MHC expression on B cells, inhibition of |
| | | monokine production |
| IL-5 | TH₂ and mast cells | eosinophil growth and function |
| IL-6 | Macrophages, fibroblasts, endothelial | IL-6 acts in synergy with IL-1 and TNF-α |
| | cells and activated T-helper cells. | in many immune responses, including T- |
| | Does not induce cytokine expression. | cell activation; primary inducer of the |
| | | acute-phase response in liver; enhances |
| | | the differentiation of B-cells and their |
| | | consequent production of |
| | | immunoglobulin; enhances |
| | | Glucocorticoid synthesis. |
| IL-7 | thymic and marrow stromal cells | T and B lymphopoiesis |
| IL-8 | Monocytes, neutrophils, macrophages, | Chemoattractant (chemokine) for |
| | and NK cells. | neutrophils, basophils and T-cells; |
| | | activates neutrophils to degranulate. |
| IL-9 | T cells | hematopoietic and thymopoietic effects |
| IL-10 | activated TH₂ cells, CD8⁺ T and B | inhibits cytokine production, promotes B |
| | cells, macrophages | cell proliferation and antibody production, |
| | | suppresses cellular immunity, mast cell |
| | | growth |
| IL-11 | stromal cells | synergisitc hematopoietic and |
| | | thrombopoietic effects |
| IL-12 | B cells, macrophages | proliferation of NK cells, INF-γ |
| | | production, promotes cell-mediated |
| | | immune functions |
| IL-13 | TH₂ cells | IL-4-like activities |
| IL-18 | macrophages/Kupffer cells, | Interferon-gamma-inducing factor with |
| | keratinocytes, glucocorticoid-secreting | potent pro-inflammatory activity |
| | adrenal cortex cells, and osteoblasts | |
| IL-21 | Activated T cells | IL21 has a role in proliferation and |
| | | maturation of natural killer (NK) cell |
| | | populations from bone marrow, in the |
| | | proliferation of mature B-cell populations |
| | | co-stimulated with anti-CD40, and in the |
| | | proliferation of T cells co-stimulated with |
| | | anti-CD3. |
| IL-23 | Activated dendritic cells | A complex of p19 and the p40 subunit of |
| | | IL-12. IL-23 binds to IL-12R beta 1 but |
| | | not IL-12R beta 2; activates Stat4 in PHA |
| | | blast T cells; induces strong proliferation |
| | | of mouse memory T cells; stimulates IFN- |
| | | gamma production and proliferation in |
| | | PHA blast T cells, as well as in CD45RO |
| | | (memory) T cells. |
| Tumor Necrosis | Primarily activated macrophages. | Once called cachectin; induces the |
| Factor | | expression of other autocrine growth |
| TNF-α | | factors, increases cellular responsiveness |
| | | to growth factors; induces signaling |
| | | pathways that lead to proliferation; |
| | | induces expression of a number of nuclear |
| | | proto-oncogenes as well as of several |
| | | interleukins. |
| (TNF-β) | T-lymphocytes, particularly cytotoxic | Also called lymphotoxin; kills a number |
| | T-lymphocytes (CTL cells); induced | of different cell types, induces terminal |
| | by IL-2 and antigen-T-Cell receptor | differentiation in others; inhibits |
| | interactions. | lipoprotein lipase present on the surface |
| | | of vascular endothelial cells. |
| Interferons | macrophages, neutrophils and some | Known as type I interferons; antiviral |
| INF-α and -β | somatic cells | effect; induction of class I MHC on all |
| | | somatic cells; activation of NK cells and |
| | | macrophages. |
| Interferon | Primarily CD8+ T-cells, activated TH₁ | Type II interferon; induces of class I |
| INF-γ | and NK cells | MHC on all somatic cells, induces class II |
| | | MHC on APCs and somatic cells, |
| | | activates macrophages, neutrophils, NK |
| | | cells, promotes cell-mediated immunity, |
| | | enhances ability of cells to present |
| | | antigens to T-cells; antiviral effects. |
| Monocyte | Peripheral blood | Attracts monocytes to sites of vascular |
| Chemoattractant | monocytes/macrophages | endothelial cell injury, implicated in |
| Protein-1 | | atherosclerosis. |
| (MCP1) | | |
| Colony | | Stimulate the proliferation of specific |
| Stimulating | | pluripotent stem cells of the bone marrow |
| Factors (CSFs) | | in adults. |
| Granulocyte- | | Specific for proliferative effects on cells |
| CSF (G-CSF) | | of the granulocyte lineage; proliferative |
| | | effects on both classes of lymphoid cells. |
| Macrophage- | | Specific for cells of the macrophage |
| CSF (M-CSF) | | lineage. |
| Granulocyte- | | Proliferative effects on cells of both the |
| MacrophageCSF | | macrophage and granulocyte lineages. |
| (GM-CSF) | | |

Other cytokines of interest that can be characterized by the invention described herein include adhesion molecules (R & D Systems, ADHESION MOLECULES I (1996), available at http://www.rndsystems.com); angiogenin (U.S. Pat. No. 4,721,672; Moener et al., 226 EUR. J. BIOCHEM. 483 (1994)); annexin V (Cookson et al., 20 GENOMICS 463 (1994); Grundmann et al., 85 PROC. NATL. ACAD. SCI. USA 3708 (1988); U.S. Pat. No. 5,767,247); caspases (U.S. Pat. No. 6,214,858; Thornberry et al., 281 SCIENCE 1312 (1998)); chemokines (U.S. Pat. Nos. 6,174,995; 6,143,289; Sallusto et al., 18 ANNU. REV. IMMUNOL. 593 (2000) Kunkel et al., 59 J. LEUKOCYTE BIOL. 81 (1996)); endothelin (U.S. Pat. Nos. 6,242,485; 5,294,569; 5,231,166); eotaxin (U.S. Pat. No. 6,271,347; Ponath et al., 97(3) J. CLIN. INVEST. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. BIOL. CHEM. 2365 (1996); Maffei et al., 92 PNAS 6957 (1995); Zhang et al. (1994) NATURE 372: 425-432); Macrophage Stimulating Protein (MSP) (U.S. Pat. Nos. 6,248,560; 6,030,949; 5,315,000); Neurotrophic Factors (U.S. Pat. Nos. 6,005,081; 5,288,622); Pleiotrophin/Midkine (PTN/MK) (Pedraza et al., 117 J. BIOCHEM. 845 (1995); Tamura et al., 3 ENDOCRINE 21 (1995); U.S. Pat. No. 5,210,026; Kadomatsu et al., 151 BIOCHEM. BIOPHYS. RES. COMMUN. 1312 (1988)); STAT proteins (U.S. Pat. Nos. 6,030,808; 6,030,780; Darnell et al., 277 SCIENCE 1630-1635 (1997)); Tumor Necrosis Factor Family (Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984)).

Also of interest regarding cytokines are proteins or chemical moieties that interact with cytokines, such as Matrix Metalloproteinases (MMPs) (U.S. Pat. No. 6,307,089; NAGASE, MATRIX METALLOPROTEINASES IN ZINC METALLOPROTEASES IN HEALTH AND DISEASE (1996)), and Nitric Oxide Synthases (NOS) (Fukuto, 34 ADV. PHARM 1 (1995); U.S. Pat. No. 5,268,465).

A further embodiment of the present invention applies the methodologies described herein to the characterization of the binding domains of blood proteins. The term “blood protein” is a generic term for a vast group of proteins generally circulating in blood plasma, and important for regulating coagulation and clot dissolution. See, e.g., Haematologic Technologies, Inc., HTI CATALOG, available at www.haemtech.com. Table 3 introduces, in a non-limiting fashion, some of the blood proteins contemplated by the present invention.

TABLE 3
Blood Proteins

| Protein | Principle Activity | Reference |
|---|---|---|
| Factor V | In coagulation, this glycoprotein pro- | Mann et al., 57 ANN. REV. BIOCHEM. |
| | cofactor, is converted to active cofactor, | 915 (1988); see also Nesheim et al., 254 |
| | factor Va, via the serine protease α- | J. BIOL. CHEM. 508 (1979); Tracy et al., |
| | thrombin, and less efficiently by its | 60 BLOOD 59 (1982); Nesheim et al., 80 |
| | serine protease cofactor Xa. The | METHODS ENZYMOL. 249 (1981); Jenny |
| | prothrombinase complex rapidly | et al., 84 PROC. NATL. ACAD. SCI. USA |
| | converts zymogen prothrombin to the | 4846 (1987). |
| | active serine protease, α-thrombin. | |
| | Down regulation of prothrombinase | |
| | complex occurs via inactivation of Va | |
| | by activated protein C. | |
| Factor VII | Single chain glycoprotein zymogen in | See generally, Broze et al., 80 METHODS |
| | its native form. Proteolytic activation | ENZYMOL. 228 (1981); Bajaj et al., 256 |
| | yields enzyme factor VIIa, which binds | J. BIOL. CHEM. 253 (1981); Williams et |
| | to integral membrane protein tissue | al., 264 J. BIOL. CHEM. 7536 (1989); |
| | factor, forming an enzyme complex that | Kisiel et al., 22 THROMBOSIS RES. 375 |
| | proteolytically converts factor X to Xa. | (1981); Seligsohn et al., 64 J. CLIN. |
| | Also known as extrinsic factor Xase | INVEST. 1056 (1979); Lawson et al., 268 |
| | complex. Conversion of VII to VIIa | J. BIOL. CHEM. 767 (1993). |
| | catalyzed by a number of proteases | |
| | including thrombin, factors IXa, Xa, | |
| | XIa, and XIIa. Rapid activation also | |
| | occurs when VII combines with tissue | |
| | factor in the presence of Ca, likely | |
| | initiated by a small amount of pre- | |
| | existing VIIa. Not readily inhibited by | |
| | antithrombin III/heparin alone, but is | |
| | inhibited when tissue factor added. | |
| Factor IX | Zymogen factor IX, a single chain | Thompson, 67 BLOOD, 565 (1986); |
| | vitamin K-dependent glycoprotein, | Hedner et al., HEMOSTASIS AND |
| | made in liver. Binds to negatively | THROMBOSIS 39-47 (R. W. Colman, J. Hirsh, |
| | charged phospholipid surfaces. | V. J. Marder, E. W. Salzman ed., |
| | Activated by factor XIα or the factor | 2^{nd} ed. J. P. Lippincott Co., Philadelphia) |
| | VIIa/tissue factor/phospholipid | 1987; Fujikawa et al., 45 METHODS IN |
| | complex. Cleavage at one site yields the | ENZYMOLOGY 74 (1974). |
| | intermediate IXα, subsequently | |
| | converted to fully active form IXaβ by | |
| | cleavage at another site. Factor IXaβ is | |
| | the catalytic component of the “intrinsic | |
| | factor Xase complex” (factor | |
| | VIIIa/IXa/Ca²⁺/phospholipid) that | |
| | proteolytically activates factor X to | |
| | factor Xa. | |
| Factor X | Vitamin K-dependent protein zymogen, | See Davie et al., 48 ADV. ENZYMOL 277 |
| | made in liver, circulates in plasma as a | (1979); Jackson, 49 ANN. REV. |
| | two chain molecule linked by a disulfide | BIOCHEM. 765 (1980); see also |
| | bond. Factor Xa (activated X) serves as | Fujikawa et al., 11 BIOCHEM. 4882 |
| | the enzyme component of | (1972); Discipio et al., 16 BIOCHEM. |
| | prothrombinase complex, responsible | 698 (1977); Discipio et al., 18 |
| | for rapid conversion of prothrombin to | BIOCHEM. 899 (1979); Jackson et al., 7 |
| | thrombin. | BIOCHEM. 4506 (1968); McMullen et |
| | | al., 22 BIOCHEM. 2875 (1983). |
| Factor XI | Liver-made glycoprotein homodimer | Thompson et al., 60 J. CLIN. INVEST. |
| | circulates, in a non-covalent complex | 1376 (1977); Kurachi et al., 16 |
| | with high molecular weight kininogen, | BIOCHEM. 5831 (1977); Bouma et al., |
| | as a zymogen, requiring proteolytic | 252 J. BIOL. CHEM. 6432 (1977); |
| | activation to acquire serine protease | Wuepper, 31 FED. PROC. 624 (1972); |
| | activity. Conversion of factor XI to | Saito et al., 50 BLOOD 377 (1977); |
| | factor XIa is catalyzed by factor XIIa. | Fujikawa et al., 25 BIOCHEM. 2417 |
| | XIa unique among the serine proteases, | (1986); Kurachi et al., 19 BIOCHEM. |
| | since it contains two active sites per | 1330 (1980); Scott et al., 69 J. CLIN. |
| | molecule. Works in the intrinsic | INVEST. 844 (1982). |
| | coagulation pathway by catalyzing | |
| | conversion of factor IX to factor IXa. | |
| | Complex form, factor XIa/HMWK, | |
| | activates factor XII to factor XIIa and | |
| | prekallikrein to kallikrein. Major | |
| | inhibitor of XIa is a₁-antitrypsin and | |
| | to lesser extent, antithrombin-III. | |
| | Lack of factor XI procoagulant activity | |
| | causes bleeding disorder: plasma | |
| | thromboplastin antecedent deficiency. | |
| Factor XII | Glycoprotein zymogen. Reciprocal | Schmaier et al., 18-38, and Davie, 242-267 |
| (Hageman | activation of XII to active serine | HEMOSTASIS & THROMBOSIS |
| Factor) | protease factor XIIa by kallikrein is | (Colman et al., eds., J. B. Lippincott Co., |
| | central to start of intrinsic coagulation | Philadelphia, 1987). |
| | pathway. Surface bound α-XIIa activates | |
| | factor XI to XIa. Secondary cleavage of | |
| | α-XIIa by kallikrein yields β-XIIa, and | |
| | catalyzes solution phase activation of | |
| | kallikrein, factor VII and the classical | |
| | complement cascade. | |
| Factor XIII | Zymogenic form of glutaminyl-peptide | See McDonaugh, 340-357 HEMOSTASIS |
| | γ-glutamyl transferase factor XIIIa | & THROMBOSIS (Colman et al., eds., |
| | (fibrinoligase, plasma transglutaminase, | J. B. Lippincott Co., Philadelphia, 1987); |
| | fibrin stabilizing factor). Made in the | Folk et al., 113 METHODS ENZYMOL. |
| | liver, found extracellularly in plasma | 364 (1985); Greenberg et al., 69 BLOOD |
| | and intracellularly in platelets, | 867 (1987). Other proteins known to be |
| | megakaryocytes, monocytes, placenta, | substrates for Factor XIIIa, that can be |
| | uterus, liver and prostrate tissues. | hemostatically important, include |
| | Circulates as a tetramer of 2 pairs of | fibronectin (Iwanaga et al., 312 ANN. |
| | nonidentical subunits (A₂B₂). Full | NY ACAD. SCI. 56 (1978)), a₂- |
| | expression of activity is achieved only | antiplasmin (Sakata et al., 65 J. CLIN. |
| | after the Ca²⁺- and fibrin(ogen)- | INVEST. 290 (1980)), collagen (Mosher |
| | dependent dissociation of B subunit | et al., 64 J. CLIN. INVEST. 781 (1979)), |
| | dimer from A₂′ dimer. Last of the | factor V (Francis et al., 261 J. BIOL. |
| | zymogens to become activated in the | CHEM. 9787 (1986)), von Willebrand |
| | coagulation cascade, the only enzyme in | Factor (Mosher et al., 64 J. CLIN. |
| | this system that is not a serine protease. | INVEST. 781 (1979)) and |
| | XIIIa stabilizes the fibrin clot by | thrombospondin (Bale et al., 260 J. |
| | crosslinking the α and γ-chains of fibrin. | BIOL. CHEM. 7502 (1985); Bohn, 20 |
| | Serves in cell proliferation in wound | MOL. CELL BIOCHEM. 67 (1978)). |
| | healing, tissue remodeling, | |
| | atherosclerosis, and tumor growth. | |
| Fibrinogen | Plasma fibrinogen, a large glycoprotein, | FURLAN, Fibrinogen, IN HUMAN |
| | disulfide linked dimer made of 3 pairs of | PROTEIN DATA, (Haeberli, ed., VCH |
| | non-identical chains (Aa, Bb and g), | Publishers, N.Y., 1995); Doolittle, in |
| | made in liver. Aa has N-terminal peptide | HAEMOSTASIS & THROMBOSIS, 491-513 |
| | (fibrinopeptide A (FPA), factor XIIIa | (3rd ed., Bloom et al., eds., Churchill |
| | crosslinking sites, and 2 phosphorylation | Livingstone, 1994); HANTGAN, et al., in |
| | sites. Bb has fibrinopeptide B (FPB), 1 | HAEMOSTASIS & THROMBOSIS 269-89 |
| | of 3 N-linked carbohydrate moieties, | (2d ed., Forbes et al., eds., Churchill |
| | and an N-terminal pyroglutamic acid. | Livingstone, 1991). |
| | The g chain contains the other N-linked | |
| | glycos. site, and factor XIIIa cross- | |
| | linking sites. Two elongated subunits | |
| | ((AaBbg)₂) align in an antiparallel way | |
| | forming a trinodular arrangement of the | |
| | 6 chains. Nodes formed by disulfide | |
| | rings between the 3 parallel chains. | |
| | Central node (n-disulfide knot, E | |
| | domain) formed by N-termini of all 6 | |
| | chains held together by 11 disulfide | |
| | bonds, contains the 2 IIa-sensitive sites. | |
| | Release of FPA by cleavage generates | |
| | Fbn I, exposing a polymerization site on | |
| | Aa chain. These sites bind to regions on | |
| | the D domain of Fbn to form proto- | |
| | fibrils. Subsequent IIa cleavage of FPB | |
| | from the Bb chain exposes additional | |
| | polymerization sites, promoting lateral | |
| | growth of Fbn network. Each of the 2 | |
| | domains between the central node and | |
| | the C-terminal nodes (domains D and E) | |
| | has parallel a-helical regions of the Aa, | |
| | Bb and g chains having protease- | |
| | (plasmin-) sensitive sites. Another major | |
| | plasmin sensitive site is in hydrophilic | |
| | preturbance of a-chain from C-terminal | |
| | node. Controlled plasmin degradation | |
| | converts Fbg into fragments D and E. | |
| Fibronectin | High molecular weight, adhesive, | Skorstengaard et al., 161 Eur. J. |
| | glycoprotein found in plasma and | BIOCHEM. 441 (1986); Kornblihtt et al., |
| | extracellular matrix in slightly different | 4 EMBO J. 1755 (1985); Odermatt et |
| | forms. Two peptide chains | al., 82 PNAS 6571 (1985); Hynes, R. O., |
| | interconnected by 2 disulfide bonds, has | ANN. REV. CELL BIOL., 1, 67 (1985); |
| | 3 different types of repeating | Mosher 35 ANN. REV. MED. 561 (1984); |
| | homologous sequence units. Mediates | Rouslahti et al., 44 Cell 517 (1986); |
| | cell attachment by interacting with cell | Hynes 48 CELL 549 (1987); Mosher 250 |
| | surface receptors and extracellular | BIOL. CHEM. 6614 (1975). |
| | matrix components. Contains an Arg- | |
| | Gly-Asp-Ser (RGDS) cell attachment- | |
| | promoting sequence, recognized by | |
| | specific cell receptors, such as those on | |
| | platelets. Fibrin-fibronectin complexes | |
| | stabilized by factor XIIIa-catalyzed | |
| | covalent cross-linking of fibronectin to | |
| | the fibrin a chain. | |
| β₂- | Also called β₂I and Apolipoprotein H. | See, e.g., Lozier et al., 81 PNAS 2640-44 |
| Glycoprotein I | Highly glycosylated single chain protein | (1984); Kato & Enjyoi 30 BIOCHEM. |
| | made in liver. Five repeating mutually | 11687-94 (1997); Wurm, 16 INT'L J. |
| | homologous domains consisting of | BIOCHEM. 511-15 (1984); Bendixen et |
| | approximately 60 amino acids disulfide | al., 31 BIOCHEM. 3611-17 (1992); |
| | bonded to form Short Consensus | Steinkasserer et al., 277 BIOCHEM. J. |
| | Repeats (SCR) or Sushi domains. | 387-91 (1991); Nimpf et al., 884 |
| | Associated with lipoproteins, binds | BIOCHEM. BIOPHYS. ACTA 142-49 |
| | anionic surfaces like anionic vesicles, | (1986); Kroll et.al. 434 BIOCHEM. |
| | platelets, DNA, mitochondria, and | BIOPHYS. Acta 490-501 (1986); Polz et |
| | heparin. Binding can inhibit contact | al., 11 INT'L J. BIOCHEM. 265-73 |
| | activation pathway in blood coagulation. | (1976); McNeil et al., 87 PNAS 4120-24 |
| | Binding to activated platelets inhibits | (1990); Galli et a;. I LANCET 1544-47 |
| | platelet associated prothrombinase and | (1990); Matsuuna et al., II LANCET 177-78 |
| | adenylate cyclase activities. Complexes | (1990); Pengo et al., 73 THROMBOSIS |
| | between b₂I and cardiolipin have been | & HAEMOSTASIS 29-34 (1995). |
| | implicated in the anti-phospholipid | |
| | related immune disorders LAC and SLE. | |
| Osteonectin | Acidic, noncollagenous glycoprotein | Villarreal et al., 28 BIOCHEM. 6483 |
| | (Mr = 29,000) originally isolated from | (1989); Tracy et al., 29 INT'L J. |
| | fetal and adult bovine bone matrix. May | BIOCHEM. 653 (1988); Romberg et al., |
| | regulate bone metabolism by binding | 25 BIOCHEM. 1176 (1986); Sage & |
| | hydroxyapatite to collagen. Identical to | Bornstein 266 J. BIOL. CHEM. 14831 |
| | human placental SPARC. An alpha | (1991); Kelm & Mann 4 J. BONE MIN. |
| | granule component of human platelets | RES. 5245 (1989); Kelm et al., 80 |
| | secreted during activation. A small | BLOOD 3112 (1992). |
| | portion of secreted osteonectin | |
| | expressed on the platelet cell surface in | |
| | an activation-dependent manner | |
| Plasminogen | Single chain glycoprotein zymogen with | See Robbins, 45 METHODS IN |
| | 24 disulfide bridges, no free sulfhydryls, | ENZYMOLOGY 257 (1976); COLLEN, |
| | and 5 regions of internal sequence | 243-258 BLOOD COAG. (Zwaal et al., |
| | homology, “kringles”, each five triple- | eds., New York, Elsevier, 1986); see |
| | looped, three disulfide bridged, and | also Castellino et al., 80 METHODS IN |
| | homologous to kringle domains in t-PA, | ENZYMOLOGY 365 (1981); Wohl et al., |
| | u-PA and prothrombin. Interaction of | 27 THROMB. RES. 523 (1982); Barlow et |
| | plasminogen with fibrin and α2- | al., 23 BIOCHEM. 2384 (1984); |
| | antiplasmin is mediated by lysine | SOTTRUP-JENSEN ET AL., 3 PROGRESS IN |
| | binding sites. Conversion of | CHEM. FIBRINOLYSIS & THROMBOLYSIS |
| | plasminogen to plasmin occurs by | 197-228 (Davidson et al., eds., Raven |
| | variety of mechanisms, including | Press, New York 1975). |
| | urinary type and tissue type | |
| | plasminogen activators, streptokinase, | |
| | staphylokinase, kallikrein, factors IXa | |
| | and XIIa, but all result in hydrolysis at | |
| | Arg560-Val561, yielding two chains | |
| | that remain covalently associated by a | |
| | disulfide bond. | |
| tissue | t-PA, a serine endopeptidase synthesized | See Plasminogen. |
| Plasminogen | by endothelial cells, is the major | |
| Activator | physiologic activator of plasminogen in | |
| | clots, catalyzing conversion of | |
| | plasminogen to plasmin by hydrolising a | |
| | specific arginine-alanine bond. Requires | |
| | fibrin for this activity, unlike the kidney- | |
| | produced version, urokinase-PA. | |
| Plasmin | See Plasminogen. Plasmin, a serine | See Plasminogen. |
| | protease, cleaves fibrin, and activates | |
| | and/or degrades compounds of | |
| | coagulation, kinin generation, and | |
| | complement systems. Inhibited by a | |
| | number of plasma protease inhibitors in | |
| | vitro. Regulation of plasmin in vivo | |
| | occurs mainly through interaction with | |
| | a₂-antiplasmin, and to a lesser extent, a₂- | |
| | macroglobulin. | |
| Platelet Factor-4 | Low molecular weight, heparin-binding | Rucinski et al., 53 BLOOD 47 (1979); |
| | protein secreted from agonist-activated | Kaplan et al., 53 BLOOD 604 (1979); |
| | platelets as a homotetramer in complex | George 76 BLOOD 859 (1990); Busch et |
| | with a high molecular weight, | al., 19 THROMB. RES. 129 (1980); Rao |
| | proteoglycan, carrier protein. Lysine- | et al., 61 BLOOD 1208 (1983); Brindley, |
| | rich, COOH-terminal region interacts | et al., 72 J. CLIN. INVEST. 1218 (1983); |
| | with cell surface expressed heparin-like | Deuel et al., 74 PNAS 2256 (1981); |
| | glycosaminoglycans on endothelial | Osterman et al., 107 BIOCHEM. |
| | cells, PF-4 neutralizes anticoagulant | BIOPHYS. RES. COMMUN. 130 (1982); |
| | activity of heparin exerts procoagulant | Capitanio et al., 839 BIOCHEM. |
| | effect, and stimulates release of | BIOPHYS. ACTA 161 (1985). |
| | histamine from basophils. Chemotactic | |
| | activity toward neutrophils and | |
| | monocytes. Binding sites on the platelet | |
| | surface have been identified and can be | |
| | important for platelet aggregation. | |
| Protein C | Vitamin K-dependent zymogen, protein | See Esmon, 10 PROGRESS IN THROMB. |
| | C, made in liver as a single chain | & HEMOSTS. 25 (1984); Stenflo, 10 |
| | polypeptide then converted to a disulfide | SEMIN. IN THROMB. & HEMOSTAS. 109 |
| | linked heterodimer. Cleaving the heavy | (1984); Griffen et al., 60 BLOOD 261 |
| | chain of human protein C converts the | (1982); Kisiel et al., 80 METHODS |
| | zymogen into the serine protease, | ENZYMOL. 320 (1981); Discipio et al., |
| | activated protein C. Cleavage catalyzed | 18 BIOCHEM. 899 (1979). |
| | by a complex of α-thrombin and | |
| | thrombomodulin. Unlike other vitamin | |
| | K dependent coagulation factors, | |
| | activated protein C is an anticoagulant | |
| | that catalyzes the proteolytic | |
| | inactivation of factors Va and VIIIa, and | |
| | contributes to the fibrinolytic response | |
| | by complex formation with plasminogen | |
| | activator inhibitors. | |
| Protein S | Single chain vitamin K-dependent | Walker, 10 SEMIN. THROMB. |
| | protein functions in coagulation and | HEMOSTAS. 131 (1984); Dahlback et al., |
| | complement cascades. Does not | 10 SEMIN. THROMB. HEMOSTAS., 139 |
| | possess the catalytic triad. Complexes | (1984); Walker 261 J. BIOL. CHEM. |
| | to C4b binding protein (C4BP) and to | 10941 (1986). |
| | negatively charged phospholipids, | |
| | concentrating C4BP at cell surfaces | |
| | following injury. Unbound S serves as | |
| | anticoagulant cofactor protein with | |
| | activated Protein C. A single cleavage | |
| | by thrombin abolishes protein S cofactor | |
| | activity by removing gla domain. | |
| Protein Z | Vitamin K-dependent, single-chain | Sejima et al., 171 BIOCHEM. |
| | protein made in the liver. Direct | BIOPHYSICS RES. COMM. 661 (1990); |
| | requirement for the binding of thrombin | Hogg et al., 266 J. BIOL. CHEM. 10953 |
| | to endothelial phospholipids. Domain | (1991); Hogg et al., 17 BIOCHEM. |
| | structure similar to that of other vitamin | BIOPHYSICS RES. COMM. 801 (1991); |
| | K-dependant zymogens like factors VII, | Han et al., 38 BIOCHEM. 11073 (1999); |
| | IX, X, and protein C. N-terminal region | Kemkes-Matthes et al., 79 THROMB. |
| | contains carboxyglutamic acid domain | RES. 49 (1995). |
| | enabling phospholipid membrane | |
| | binding. C-terminal region lacks | |
| | “typical” serine protease activation site. | |
| | Cofactor for inhibition of coagulation | |
| | factor Xa by serpin called protein Z- | |
| | dependant protease inhibitor. Patients | |
| | diagnosed with protein Z deficiency | |
| | have abnormal bleeding diathesis during | |
| | and after surgical events. | |
| Prothrombin | Vitamin K-dependent, single-chain | Mann et al., 45 METHODS IN |
| | protein made in the liver. Binds to | ENZYMOLOGY 156 (1976); Magnusson |
| | negatively charged phospholipid | et al., PROTEASES IN BIOLOGICAL |
| | membranes. Contains two “kringle” | CONTROL 123-149 (Reich et al., eds. |
| | structures. Mature protein circulates in | Cold Spring Harbor Labs., New York |
| | plasma as a zymogen and, during | 1975); Discipio et al., 18 BIOCHEM. 899 |
| | coagulation, is proteolytically activated | (1979). |
| | to the potent serine protease α-thrombin. | |
| α-Thrombin | See Prothrombin. During coagulation, | 45 METHODS ENZYMOL. 156 (1976). |
| | thrombin cleaves fibrinogen to form | |
| | fibrin, the terminal proteolytic step in | |
| | coagulation, forming the fibrin clot. | |
| | Thrombin also responsible for feedback | |
| | activation of procofactors V and VIII. | |
| | Activates factor XIII and platelets, | |
| | functions as vasoconstrictor protein. | |
| | Procoagulant activity arrested by | |
| | heparin cofactor II or the antithrombin | |
| | III/heparin complex, or complex | |
| | formation with thrombomodulin. | |
| | Formation of thrombin/thrombomodulin | |
| | complex results in inability of thrombin | |
| | to cleave fibrinogen and activate factors | |
| | V and VIII, but increases the efficiency | |
| | of thrombin for activation of the | |
| | anticoagulant, protein C. | |
| β-Thromboglobulin | Low molecular weight, heparin-binding, | See, e.g., George 76 BLOOD 859 (1990); |
| | platelet-derived tetramer protein, | Holt & Niewiarowski 632 BIOCHIM. |
| | consisting of four identical peptide | BIOPHYS. ACTA 284 (1980); |
| | chains. Lower affinity for heparin than | Niewiarowski et al., 55 BLOOD 453 |
| | PF-4. Chemotactic activity for human | (1980); Varma et al., 701 BIOCHIM. |
| | fibroblasts, other functions unknown. | BIOPHYS. ACTA 7 (1982); Senior et al., |
| | | 96 J. CELL. BIOL. 382 (1983). |
| Thrombopoietin | Human TPO (Thrombopoietin, Mpl- | Horikawa et al., 90(10) BLOOD 4031-38 |
| | ligand, MGDF) stimulates the | (1997); de Sauvage et al., 369 NATURE |
| | proliferation and maturation of | 533-58 (1995). |
| | megakaryocytes and promotes increased | |
| | circulating levels of platelets in vivo. | |
| | Binds to c-Mpl receptor. | |
| Thrombospondin | High-molecular weight, heparin-binding | Dawes et al., 29 THROMB. RES. 569 |
| | glycoprotein constituent of platelets, | (1983); Switalska et al., 106 J. LAB. |
| | consisting of three, identical, disulfide- | CLIN MED. 690 (1985); Lawler et al. |
| | linked polypeptide chains. Binds to | 260 J. BIOL. CHEM. 3762 (1985); Wolff |
| | surface of resting and activated platelets, | et al., 261 J. BIOL. CHEM. 6840 (1986); |
| | may effect platelet adherence and | Asch et al., 79 J. CLIN. CHEM. 1054 |
| | aggregation. An integral component of | (1987); Jaffe et al., 295 NATURE 246 |
| | basement membrane in different tissues. | (1982); Wright et al., 33 J. HISTOCHEM. |
| | Interacts with a variety of extracellular | CYTOCHEM. 295 (1985); Dixit et al., |
| | macromolecules including heparin, | 259 J. BIOL. CHEM. 10100 (1984); |
| | collagen, fibrinogen and fibronectin, | Mumby et al., 98 J. CELL. BIOL. 646 |
| | plasminogen, plasminogen activator, | (1984); Lahav et al, 145 EUR. J. |
| | and osteonectin. May modulate cell- | BIOCHEM. 151 (1984); Silverstein et al, |
| | matrix interactions. | 260 J. BIOL. CHEM. 10346 (1985); |
| | | Clezardin et al. 175 EUR. J. BIOCHEM. |
| | | 275 (1988); Sage & Bornstein (1991). |
| Von Willebrand | Multimeric plasma glycoprotein made of | Hoyer 58 BLOOD 1 (1981); Ruggeri & |
| Factor | identical subunits held together by | Zimmerman 65 J. CLIN. INVEST. 1318 |
| | disulfide bonds. During normal | (1980); Hoyer & Shainoff 55 BLOOD |
| | hemostasis, larger multimers of vWF | 1056 (1980); Meyer et al., 95 J. LAB. |
| | cause platelet plug formation by forming | CLIN. INVEST. 590 (1980); Santoro 21 |
| | a bridge between platelet glycoprotein | THROMB. RES. 689 (1981); Santoro, & |
| | IB and exposed collagen in the | Cowan 2 COLLAGEN RELAT. RES. 31 |
| | subendothelium. Also binds and | (1982); Morton et al., 32 THROMB. RES. |
| | transports factor VIII (antihemophilic | 545 (1983); Tuddenham et al., 52 BRIT. |
| | factor) in plasma. | J. HAEMATOL. 259 (1982). |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin, Troponin I, Troponin T, Prostatic Acid Phosphatase, Retinol Binding Globulin (RBP). All of these proteins, and sources thereof, are known in the art. Many of these proteins are available commercially from, for example, Research Diagnostics, Inc. (Flanders, N.J.).

Another embodiment applies the methodologies of the present invention to the characterization of the binding domain of a neurotransmitter or the receptor of a neurotransmitter. Neurotransmitters are chemicals, some of them proteinaceous, made by neurons and used by them to transmit signals to the other neurons or non-neuronal cells (e.g., skeletal muscle, myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See generally COOPER, BLOOM & ROTH, THE BIOCHEM. BASIS OF NEUROPHARMACOLOGY (7th Ed. Oxford Univ. Press, NYC, 1996); http://web.indstate.edu/thcme/mwking/nerves. Neurotransmitters contemplated in the present invention include, but are not limited to, Acetylcholine, Serotonin, γ-aminobutyrate (GABA), Glutamate, Aspartate, Glycine, Histamine, Epinephrine, Norepinephrine, Dopamine, Adenosine, ATP, Nitric oxide, and any of the peptide neurotransmitters such as those derived from pre-opiomelanocortin (POMC), as well as antagonists and agonists thereof.

The binding domains of numerous other proteins or peptides can be characterized by the methods of the present invention. Table 4 presents a non-limiting list and description of some pharmacologically active peptides which can be incorporated into the methods contemplated by the present invention.

TABLE 4
Pharmacologically active peptides

| Binding partner/ | | |
|---|---|---|
| Protein of interest | | |
| (form of peptide) | Pharmacological activity | Reference |
| EPO receptor | EPO mimetic | Wrighton et al., 273 SCIENCE 458-63 |
| (intrapeptide | | (1996); U.S. Pat. No. 5,773,569, issued |
| disulfide-bonded) | | Jun. 30, 1998. |
| EPO receptor | EPO mimetic | Livnah et al., 273 SCIENCE 464-71 |
| (C-terminally cross- | | (1996); Wrighton et al., 15 NATURE |
| linked dimer) | | BIOTECHNOLOGY 1261-5 (1997); Int'l |
| | | Patent Application WO 96/40772, |
| | | published Dec. 19, 1996. |
| EPO receptor | EPO mimetic | Naranda et al., 96 PNAS 7569-74 (1999). |
| (linear) | | |
| c-Mpl | TPO-mimetic | Cwirla et al., 276 SCIENCE 1696-9 (1997); |
| (linear) | | U.S. Pat. No. 5,869,451, issued Feb. |
| | | 9,1999; U.S. Pat. No. 5,932,946, issued |
| | | Aug. 3,1999. |
| c-Mpl | TPO-mimetic | Cwirla et al., 276 SCIENCE 1696-9 (1997). |
| (C-terminally cross- | | |
| linked dimer) | | |
| (disulfide-linked | stimulation of | Paukovits et al., 364 HOPPE-SEYLERS Z. |
| dimer) | hematopoesis | PHYSIOL. CHEM. 30311 (1984); |
| | (“G-CSF-mimetic”) | Laerurngal., 16 Exp. HEMAT. 274-80 |
| | | (1988). |
| (alkylene-linked dimer) | G-CSF-mimetic | Batnagar et al., 39 J. MED. CHEM. 38149 |
| | | (1996); Cuthbertson et al., 40 J. MED. |
| | | CHEM. 2876-82 (1997); King et al., 19 |
| | | Exp. HEMATOL. 481 (1991); King et al., |
| | | 86(Suppl. 1) BLOOD 309 (1995). |
| IL-1 receptor | inflammatory and | U.S. Pat. No. 5,608,035; U.S. Pat. No. |
| (linear) | autoimmune diseases (“IL-1 | 5,786,331; U.S Pat. No. 5,880,096; |
| | antagonist” or “IL-1 ra- | Yanofsky et al., 93 PNAS 7381-6 (1996); |
| | mimetic”) | Akeson et al., 271 J. BIOL. CHEM. 30517-23 |
| | | (1996); Wiekzorek et al., 49 POL. J. PHARMACOL. |
| | | 107-17 (1997); Yanofsky, |
| | | 93 PNAS 7381-7386 (1996). |
| Facteur thyrnique | stimulation of lymphocytes | Inagaki-Ohara et al., 171 CELLULAR |
| (linear) | (FTS-mimetic) | IMMUNOL. 30-40 (1996); Yoshida, 6 J. |
| | | IMMUNOPHARMACOL 141-6 (1984). |
| CTLA4 MAb | CTLA4-mimetic | Fukumoto et al., 16 NATURE BIOTECH. |
| (intrapeptide di-sulfide | | 267-70 (1998). |
| bonded) | | |
| TNF-α receptor | TNF-α antagonist | Takasaki et al., 15 NATURE BIOTECH. |
| (exo-cyclic) | | 1266-70 (1997); WO 98/53842, published |
| | | Dec. 3, 1998. |
| TNF-α receptor | TNF-α antagonist | Chirinos-Rojas, J. IMM., 5621-26. |
| (linear) | | |
| C3b | inhibition of complement | Sahu et al., 157 IMMUNOL. 884-91 (1996); |
| (intrapeptide di-sulfide | activation; autoimmune | Morikis et al., 7 PROTEIN SCI. 6 19-27 |
| bonded) | diseases (C3b antagonist) | (1998). |
| vinculin | cell adhesion processes, cell | Adey et al., 324 BIOCHEM. J. 523-8 |
| (linear) | growth, differentiation | (1997). |
| | wound healing, tumor | |
| | metastasis (“vinculin | |
| | binding”) | |
| C4 binding protein (C413P) | anti-thrombotic | Linse et al. 272 BIOL. CHEM. 14658-65 |
| (linear) | | (1997). |
| urokinase receptor | processes associated with | Goodson et al., 91 PNAS 7129-33 (1994); |
| (linear) | urokinase interaction with its | International patent application WO |
| | receptor (e.g. angiogenesis, | 97/35969, published Oct. 2, 1997. |
| | tumor cell invasion and | |
| | metastasis; (URK antagonist | |
| Mdm2, Hdm2 | Inhibition of inactivation of | Picksley et al., 9 ONCOGENE 2523-9 |
| (linear) | p53 mediated by Mdm2 or | (1994); Bottger et al. 269 J. MOL. BIOL. |
| | hdm2; anti-tumor | 744-56 (1997); Bottger et al., 13 |
| | (“Mdm/hdm antagonist”) | ONCOGENE 13: 2141-7 (1996). |
| p21^{WAFI} | anti-tumor by mimicking the | Ball et al., 7 CURR. BIOL. 71-80 (1997). |
| (linear) | activity of p21^{WAFI} | |
| farnesyl transferase | anti-cancer by preventing | Gibbs et al., 77 CELL 175-178 (1994). |
| (linear) | activation of ras oncogene | |
| Ras effector domain | anti-cancer by inhibiting | Moodie et at., 10 TRENDS GENEL 44-48 |
| (linear) | biological function of the ras | (1994); Rodriguez et al., 370 NATURE |
| | oncogene | 527-532 (1994). |
| SH2/SH3 domains | anti-cancer by inhibiting | Pawson et al, 3 CURR. BIOL. 434-432 |
| (linear) | tumor growth with activated | (1993); Yu et al., 76 CELL 933-945 |
| | tyrosine kinases | (1994). |
| p16^{INK4} | anti-cancer by mimicking | Fahraeus et al., 6 CURR. BIOL. 84-91 |
| (linear) | activity of p16; e.g., | (1996). |
| | inhibiting cyclin D-Cdk | |
| | complex (“p, 16-mimetic”) | |
| Src, Lyn | inhibition of Mast cell | Stauffer et al., 36 BIOCHEM. 9388-94 |
| (linear) | activation, IgE-related | (1997). |
| | conditions, type I | |
| | hypersensitivity (“Mast cell | |
| | antagonist”). | |
| Mast cell protease | treatment of inflammatory | International patent application WO |
| (linear) | disorders mediated by | 98/33812, published Aug. 6, 1998. |
| | release of tryptase-6 (“Mast | |
| | cell protease inhibitors”) | |
| SH3 domains | treatment of SH3-mediated | Rickles et al., 13 EMBO J. 5598-5604 |
| (linear) | disease states (“SH3 | (1994); Sparks et al., 269 J. |
| | antagonist”) | BIOL. CHEM. 238536 (1994); |
| | | Sparks et al., 93 PNAS 1540-44 |
| | | (1996). |
| HBV core antigen (HBcAg) | treatment of HBV viral | Dyson & Muray, PNAS 2194-98 |
| (linear) | antigen (HBcAg) infections | (1995). |
| | (“anti-HBV”) | |
| selectins | neutrophil adhesion | Martens et al., 270 J. BIOL. CHEM. |
| (linear) | inflammatory diseases | 21129-36 (1995); |
| | (“selectin antagonist”) | European Pat. App. EP 0 714 |
| | | 912, published Jun. 5, 1996. |
| calmodulin | calmodulin | Pierce et al, 1 MOLEC. |
| (linear, cyclized) | antagonist | DIVEMILY 25965 (1995); |
| | | Dedman et al., 267 J. BIOL. CHEM. |
| | | 23025-30 (1993); Adey |
| | | & Kay, 169 GENE 133-34 |
| | | (1996). |
| integrins | tumor-homing; treatment for | International patent applications WO |
| (linear, cyclized) | conditions related to | 95/14714, published Jun. 1, 1995; WO |
| | integrin-mediated cellular | 97/08203, published Mar. 6,1997; WO |
| | events, including platelet | 98/10795, published Mar. 19,1998; WO |
| | aggregation, thrombosis, | 99/24462, published May 20, 1999; Kraft |
| | wound healing, osteoporosis, | et al., 274 J. BIOL. CHEM. 1979-85 (1999) |
| | tissue repair, angiogenesis | |
| | (e.g., for treatment of cancer) | |
| | and tumor invasion | |
| | (“integrin-binding”) | |
| fibronectin and extracellular | treatment of inflammatory | International patent application WO |
| matrix components of T-cells | and autoimmune conditions | 98/09985, published Mar. 12, 1998. |
| and macrophages | | |
| (cyclic, linear) | | |
| somatostatin and cortistatin | treatment or prevention of | European patent application EP 0 911 |
| (linear) | hormone-producing tumors, | 393, published Apr. 28, 1999. |
| | acromegaly, giantism, | |
| | dementia, gastric ulcer, | |
| | tumor growth, inhibition of | |
| | hormone secretion, | |
| | modulation of sleep or | |
| | neural activity | |
| bacterial lipopoly-saccharide | antibiotic; septic shock; | U.S. Pat. No. 5,877,151, issued Mar. 2, |
| (linear) | disorders modulatable by | 1999. |
| | CAP37 | |
| parclaxin, mellitin | antipathogenic | International patent application WO |
| (linear or cyclic) | | 97/31019, published 28 Aug. 1997. |
| VIP | impotence, neuro- | International patent application WO |
| (linear, cyclic) | degenerative disorders | 97/40070, published Oct. 30, 1997. |
| CTLs | cancer | European patent application EP 0 770 |
| (linear) | | 624, published May 2, 1997. |
| THF-gamma2 | | Burnstein, 27 BIOCHEM. 4066-71 (1988). |
| (linear) | | |
| Amylin | | Cooper, 84 PNAS 8628-32 (1987). |
| (linear) | | |
| Adreno-medullin | | Kitamura, 192 BBRC 553-60 (1993). |
| (linear) | | |
| VEGF | anti-angiogenic; cancer, | Fairbrother, 37 BIOCHEM. 17754-64 |
| (cyclic, linear) | rheumatoid arthritis, diabetic | (1998). |
| | retinopathy, psoriasis | |
| | (“VEGF antagonist”) | |
| MMP | inflammation and | Koivunen, 17 NATURE BIOTECH. 768-74 |
| (cyclic) | autoimmune disorders; | (1999). |
| | tumor growth (“MMP | |
| | inhibitor”) | |
| HGH fragment | | U.S. Pat. No. 5,869,452, issued |
| (linear) | | Feb. 9, 1999. |
| Echistatin | inhibition of platelet | Gan, 263 J. BIOL. 19827-32 (1988). |
| | aggregation | |
| SLE autoantibody | SLE | International patent application WO |
| (linear) | | 96/30057, published Oct. 3, 1996. |
| GD1 alpha | suppression of tumor | Ishikawa et al., 1 FEBS LETT. 20-4 |
| | metastasis | (1998). |
| anti-phospholipid β-2 | endothelial cell activation, | Blank Mal., 96 PNAS 5164-8 (1999). |
| glycoprotein-1 (β2GPI) | anti-phospholipid syndrome | |
| antibodies | (APS), thromboembolic | |
| | phenomena, | |
| | thrombocytopenia, and | |
| | recurrent fetal loss | |
| T-Cell Receptor β chain | diabetes | International patent application WO |
| (linear) | | 96/101214, published Apr. 18, 1996. |

Regarding a particular embodiment of the present invention, there are two pivotal cytokines in the pathogenesis of rheumatoid arthritis, IL-1 and TNF-α. They act synergistically to induce each other, other cytokines, and COX-2. Research suggests that IL-1 is a primary mediator of bone and cartilage destruction in rheumatoid arthritis patients, whereas TNF-α appears to be the primary mediator of inflammation.

Thus, an embodiment of the present invention applies the methodologies described herein to characterizing the binding domain of an anti-TNF-α antibody. Anti-TNF-α antibodies have shown great promise as therapeutics. For example, Infliximab, provided commercially as REMICADE® by Centocor, Inc. (Malvern, Pa.) has been used for the treatment of several chronic autoimmune diseases such as Crohn's disease and rheumatoid arthritis. Treacy, 19(4) HUM. EXP. TOXICOL. 226-28 (2000); see also Chantry, 2(1) CURR. OPIN. ANTI-INFLAMMATORY IMMUNOMODULATORY INVEST. DRUGS 31-34 (2000); Rankin et al., 34(4) BRIT. J. RHEUMATOLOGY 334-42 (1995). Alternatively, murine-derived anti-TNF-α antibodies can be utilized as ligands in the methodologies of the present invention. Saravolatz et al., 169(1) J. INFECT. DIS. 214-17 (1994).

Alternatively, a type of binding domain that can be characterized by the methodologies of the present invention can be a binding domain for or of a thrombolytic. For example, the thrombolytic can be tPA, or a functional variation thereof. RETAVASE®, produced by Centocor, Inc. (Malvern, Pa.), is a variant tPA with a prolonged half-life. Interestingly, in mice, the combination of Retavase and the IIb/IIIa receptor antagonist 7E3F(ab′)₂ markedly augmented the dissolution of pulmonary embolism. See U.S. Provisional Patent Application Serial No. 60/304409.

Another type of binding domain that can be characterized by the methodologies of the present invention can be all or part of a TNF receptor, or protein or peptide that interacts with such a receptor. For example, Etanercept is a dimeric fusion protein consisting of the extracellular ligand-binding portion of the human 75 kilodalton (p75) tumor necrosis factor receptor (TNFR) linked to the Fe portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept can be obtained as ENBREL™, manufactured by Immunex Corp. (Seattle, Wash.). See also Hughes et al., (6) BIODRUGS 379-93 (2001).

Another example of a human TNF receptor exists as well, identified as p55. Kalinkovich et al., J. INFERON & CYTOKINE RES. 15749-57 (1995). This receptor has also been explored for use in therapy. See, e.g., Qian et al., 118 ΛRCH. OPHTHALMOL. 1666-71 (2000). A previous formulation of the soluble p55 TNF receptor had been coupled to polyethylene glycol [r-metHuTNFbp PEGylated dimer (TNFbp)], and demonstrated clinical efficacy but was not suitable for a chronic indication due to the development antibodies upon multiple dosing, which resulted in increased clearance of the drug. A second generation molecule was designed to remove the antigenic epitopes of TNFbp, and can be useful in treating patients with rheumatoid arthritis. Davis et al., Presented at the Ann. European Cong. Rheumatology, Nice, France (Jun. 21-24, 2000).

Another embodiment of the present invention applies the methodologies described herein to the characterization of an IL-1 receptor antagonist (IL-1Ra), a naturally occurring cytokine antagonist that demonstrates anti-inflammatory properties by balancing the destructive effects of IL-1α and IL-1β, in rheumatoid arthritis but does not induce any intracellular response. Hence, in a preferred embodiment of the invention, the methodologies comprise IL-1Ra, or any structural or functional analog thereof. Two structural variants of IL-1Ra exist: a 17-kDa form that is secreted from monocytes, macrophages, neutrophils, and other cells (sIL-1Ra) and an 18-kDa form that remains in the cytoplasm of keratinocytes and other epithelial cells, monocytes, and fibroblasts (icIL-1Ra). An additional 16-kDa intracellular isoform of IL-1Ra exists in neutrophils, monocytes, and hepatic cells. Both of the major isoforms of IL-1Ra are transcribed from the same gene through the use of alternative first exons. The production of IL-1Ra is stimulated by many substances including adherent IgG, other cytokines, and bacterial or viral components. The tissue distribution of IL-1Ra in mice indicates that sIL-1Ra is found predominantly in peripheral blood cells, lungs, spleen, and liver, while icIL-1Ra is found in large amounts in skin. Studies in transgenic and knockout mice indicate that IL-1Ra is important in host defense against endotoxin-induced injury. IL-1Ra is produced by hepatic cells with the characteristics of an acute phase protein. Endogenous IL-1Ra is produced in human autoimmune and chronic inflammatory diseases. The use of neutralizing anti-IL-1Ra antibodies has demonstrated that endogenous IL-1Ra is an important natural antiinflammatory protein in arthritis, colitis, and granulomatous pulmonary disease. Patients with rheumatoid arthritis treated with IL-1Ra for six months exhibited improvements in clinical parameters and in radiographic evidence of joint damage. Arend et al., 16 ANN. REV. IMMUNOL. 27-55 (1998).

Yet another example of an IL-1Ra that can be characterized by the methods of the present invention is a recombinant human version called interleukin-1 17.3 Kd met-IL1ra, or Anakinra, produced by Amgen, (San Francisco, Calif.) under the name KINERET™. Anakinra has also shown promise in clinical studies involving patients with rheumatoid arthritis. Presented at the 65th Ann. Sci. Meeting of Am. College Rheumatology (Nov. 12, 2001).

Another method of the present invention incorporates a moiety that targets cyclooxigenase-2 (COX-2). COX-2 selective inhibitors-such as valdecoxib, etoricoxib, celecoxib and rofecoxib are less toxic to the gastrointestinal (GI) tract than conventional nonsteroidal anti-inflammatory drugs (NSAIDs), while possessing equivalent analgesic efficacy for conditions such as osteoarthritis (OA), rheumatoid arthritis (RA), dental pain and menstrual pain.

The methodologies of the present invention may also be applied to characterize a selective p38 Mitogen-Activated Protein Kinase (p38 MAP kinase) inhibitor. For example, the compound SB 242235 is a potent and selective p38 MAP kinase inhibitor. The compound is active in the adjuvant arthritic rat, where it inhibits inflammation and has significant joint-protective effects as measured by changes in bone mineral density, magnetic resonance imaging, micro-computed tomography, and histology. These studies indicate that cytokine-suppressing, low molecular weight p38 inhibitors can be orally active, disease-modifying agents in the treatment of rheumatoid arthritis. Badger et al, Disease-Modifying Activity of SB 242235, A Selective Inhibitor of p38 Mitogen-Activated Protein Kinase, in Rat Adjuvant-Induced Arthritis, Proceedings of the 1999 AACR, NCI, EORTC Int'l Conference, Am. Assoc. for Cancer Res.

The methodologies of the present invention may also be applied to characterize the binding domain associated with interleukin 12 (IL-12), a heterodimeric cytokine consisting of glycosylated polypeptide chains of 35 and 40 kD which are disulfide bonded. The cytokine is synthesized and secreted by antigen presenting cells, including dendritic cells, monocytes, macrophages, B cells, Langerhans cells and keratinocytes, as well as natural killer (NK) cells. IL-12 mediates a variety of biological processes and has been referred to as NK cell stimulatory factor (NKSF), T-cell stimulating factor, cytotoxic T-lymphocyte maturation factor and EBV-transformed B-cell line factor. Curfs et al., 10 CLIN. MICRO. REV. 742-80 (1997). Interleukin-12 can bind to the IL-12 receptor expressed on the plasma membrane of cells (e.g., T-cells, NK cells), thereby altering (e.g., initiating, preventing) biological processes. For example, the binding of IL-12 to the IL-12 receptor can stimulate the proliferation of pre-activated T-cells and NK cells, enhance the cytolytic activity of cytotoxic T-cells (CTL), NK cells and LAK (lymphokine activated killer) cells, induce production of gamma interferon (IFN GAMMA) by T-cells and NK cells and induce differentiation of naive Th0 cells into Th1 cells that produce IFN GAMMA and IL-2. Trinchieri, 13 ANN. REV. IMMUNOLOGY 251-76 (1995). In particular, IL-12 is vital for the generation of cytolytic cells (e.g., NK, CTL) and for mounting a cellular immune response (e.g., a Th1 cell mediated immune response). Thus, IL-12 is critically important in the generation and regulation of both protective immunity (e.g., eradication of infections) and pathological immune responses (e.g., autoimmunity). Hendrzak et al., 72 LAB. INVESTIGATION 619-37 (1995). Accordingly, an immune response (e.g., protective or pathogenic) can be enhanced, suppressed or prevented by manipulation of the biological activity of IL-12 in vivo, for example, by means of an antibody.

The methodologies of the present invention may also be applied to characterize the binding domain of an integrin or integrin-binding receptor. Integrins have been implicated in the angiogenic process, by which tumor cells form new blood vessels that provide tumors with nutrients and oxygen, carry away waste products, and to act as conduits for the metastasis of tumor cells to distant sites. Gastl et al., 54 ONCOL. 177-84 (1997). Integrins are heterodimeric transmembrane proteins that play critical roles in cell adhesion to the extracellular matrix (ECM) which, in turn, mediates cell survival, proliferation and migration through intracellular signaling. During angiogenesis, a number of integrins that are expressed on the surface of activated endothelial cells regulate critical adhesive interactions with a variety of ECM proteins to regulate distinct biological events such as cell migration, proliferation and differentiation. Specifically, the closely related but distinct integrins αVβ3 and αVβ5 have been shown to mediate independent pathways in the angiogenic process. An antibody generated against αVβ3 blocked basic fibroblast growth factor (bFGF) induced angiogenesis, whereas an antibody specific to αVβ5 inhibited vascular endothelial growth factor-induced (VEGF-induced) angiogenesis. Eliceiri et al., 103 J. CLIN. INVEST. 1227-30 (1999); Friedlander et al., 270 SCIENCE 1500-02 (1995). Such integrins may include, but are not limited to α1, α2, α3, α4, α5, α6, α7, α8, α9, αD, αL, αM, αV, αX, αIIb, αIELb, β1, β2, β3, β4, β5, β6, β7, β8, α1β1, α2β1, α3β1, α4β1, α5β1, α6β1, α7β1, α8β1, α9β1, α4β7, α6β4, αDβ2, αLβ2, αMβ2, αVβ1, αVβ3, αVβ5, αVβ6, αVβ8, αXβ2, αIIbβ3, αIELbβ7, and any structural or functional analog thereof.

In another preferred embodiment of the invention, the methodologies of the present invention can be used to characterize the binding domain of a biomolecule that serves as a glycoprotein IIb/IIIa receptor antagonist. The IIb/IIIa receptor antagonist can be, but is not limited to, an antibody, a fragment of an antibody, a peptide, or an organic molecule. See, e.g., U.S. Pat. Nos. 5,976,532, 5,877,006, 5,770,198; Coller, 78 THROM HAEMOST. 730-35 (1997); JORDAN ET AL., in ADHESION RECEPTORS AS THERAPEUTIC TARGETS 281-305 (Horton, ed. CRC Press, New York, 1996); Jordan et al., in NEW THERAPEUTIC AGENTS IN THROMBOSIS & THROMBOLYSIS (Sasahara & Loscalzo, eds. Marcel Kekker, Inc. New York, 1997).

As noted previously, the techniques of the present invention can be used to characterize the binding domains of receptors, or fragments thereof, and activated receptors, i.e., peptides associated with their corresponding receptors, or fragments thereof. These complexes may mimic activated receptors and thus affect a particular biological activity. Alternatively, the receptor can be genetically re-engineered to adopt the activated conformation. For example, the thrombin-bound conformation of fibrinopeptide A exhibits a strand-turn-strand motif, with a -turn centered at residues Glu-11 and Gly-12. Molecular modeling analysis indicates that the published fibrinopeptide conformation cannot bind reasonably to thrombin, but that reorientation of two residues by alignment with bovine pancreatic trypsin inhibitor provides a good fit within the deep thrombin cleft and satisfies all of the experimental nuclear Overhauser effect data. Based on this analysis, a researchers were able to successfully design and synthesize hybrid peptide mimetic substrates and inhibitors that mimic the proposed β-turn structure. The results indicate that the turn conformation is an important aspect of thrombin specificity, and that the turn mimetic design successfully mimics the thrombin-bound conformation of fibrinopeptide. Nakanishi et al., 89(5) PNAS 1705-09 (1992).

Another example of activated-receptor moieties that can be characterized by the present invention concerns the peptido mimetics of the erythropoietin (Epo) receptor. By way of background, the binding of Epo to the Epo receptor (EpoR) is crucial for production of mature, red blood cells. The Epo-bound, actvated EpoR is a dimer. See, e.g., Constantinescu et al., 98 PNAS 4379-84 (2001). In its natural state, the first EpoR in the dimer binds Epo with a high affinity whereas the second EpoR molecule binds to the complex with a low affinity. Bivalent anti-EpoR antibodies have been reported to activate EopR, probably by dimerization of the EpoR. Additionally, small synthetic peptides, that do not have any sequence homology with the Epo molecule, are also able to mimic the biologic effects of Epo but with a lower affinity. Their mechanism of action is probably also based on the capacity to produce dimerization of the EpoR. Hence, an embodiment of the present invention provides for a methodology involving an activated EpoR mimetic. Indeed, a particularly useful application of the present invention allows for the construction of peptido mimetics based on the information obtained from the NMR characterization described herein.

The methodologies contemplated by the present invention may also be used to identify the binding domains associated with antimicrobial agents or portions thereof, which include antibacterial agents, antiviral agents, antifungal agents, antimycobacterial agents, and antiparasitic agents. Such binding domains can be part of the antimicrobial agents or the proteins or receptors to which such agents bind. In one embodiment of the present invention, either an agent or a protein/receptor can be isotopically labeled. Antibacterials include, but are not limited to, Beta-lactams (such as Penicillins and Cephalosporins), Aminoglycosides (such as Gentamicin), Macrolides (such as Erythromycin), Fluoroquinolones, Metronidazole, Sulfonamides, Tetracyclines, Trimethroprim, and Vancomycin. Antifungal agents include, but are not limited to Amphotericin, Fluconazole, Flucytosine, Itraconazole, and Ketoconazole. Antiparasitic agents include, but are not limited to, Ivermectin, Mebendazole, Mefloquine, Pentamidine, Praziquantel, Pyrimethamine, and Quinine. Antiviral agents include, but are not limited to, Acyclovir, Amantadine, Didanosine, Famciclovir, Foscarnet, Ganciclovir, Rimatandine, Stavudine, Zalcitabine, and Zidovudine. Antimycobacterial agents include, but are not limited to, Isoniazid, Rifampin, Streptomycin, Dapsone. SANFORD ET AL., GUIDE TO ANTIMICROBIAL THERAPY (25th ed., Antimicrobial Therapy, Inc., Dallas, Tex. 1995).

In another embodiment of the invention, the NMR methodologies may identify the binding domain of a cell cycle protein. Cell cycle proteins are known in the art, and include cyclins, such as G₁ cyclins, S-phase cyclins, M-phase cyclins, cyclin A, cyclin D and cyclin E; the cyclin-dependent kinases (CDKs), such as G₁ CDKs, S-phase CDKs and M-phase CDKs, CDK2, CDK4 and CDK 6; and the tumor suppressor genes such as Rb and p53. Cell cycle proteins also include those proteins involved in apoptosis, such as Bcl-2 and caspase proteins; proteins associated with Cdc42 signaling, p70 S6 kinase and PAK regulation; and integrins, discussed elsewhere. Also included in the cell cycle proteins of the present invention are anaphase-promoting complex (APC) and other proteolytic enzymes. The APC triggers the events leading to destruction of the cohesins and thus allowing sister chromatids to separate, and degrades the mitotic (M-phase) cyclins. Other relevant cell cycle proteins include S-phase promoting factor, M-phase promoting factor that activates APC. Kimball, Kimball's Biology Pages, at http://www.ultranet.com/˜jkimball/BiologyPages. Cell cycle proteins also include p13, p27, p34, p60, p80, histone H1, centrosomal proteins, lamins, and CDK inhibitors.

As noted previously, the NMR methodologies of the present invention can be useful to identify the epitope of a particular antigen. Antigens, in a broad sense, may include any molecule to which an antibody, or functional fragment thereof, binds. Such antigens can be pathogen derived, and be associated with either MHC class I or MHC class II reactions. These antigens can be proteinaceous. These antigens may also be non-proteinaceous, for example in instances where the antigens comprise non-identical, repeating units such as, for example, carbohydrate units. Carbohydrate antigens are present on cell surfaces of all types of cells, including normal human blood cells and foreign, bacterial cell walls or viral membranes. Nucleic acids may also be antigenic when associated with proteins, and are hence included within the scope of antigens encompassed in the present invention. See SEARS, IMMUNOLOGY (W. H. Freeman & Co. and Sumanas, Inc., 1997), available online at http://www.whfreeman.com/immunology.

For example, antigens can be derived from a pathogen, such as a virus, bacterium, mycoplasm, fungus, parasite, or from another foreign substance, such as a toxin. Such bacterial antigens may include or be derived from Bacillus anthracis, Bacillus tetani, Bordetella pertusis; Brucella spp., Corynebacterium diphtheriae, Clostridium botulinum, Clostridium perfringens, Coxiella burnetii, Francisella tularensis, Mycobacterium leprae, Mycobacterium tuberculosis, Salmonella typhimurium, Streptocccus pneumoniae, Escherichia coli, Haemophilus influenzae, Shigella spp., Staphylococcus aureus, Neisseria gonorrhoeae, Neisseria meningiditis, Treponema pallidum, Yersinia pestis, Vibrio cholerae. Often, the oligosaccharide structures of the outer cell walls of these microbes afford superior protective immunity, but must be conjugated to an appropriate carrier for that effect.

Viruses and viral antigens that can be involved with the methodologies of the current invention include, but are not limited to, HBeAg, Hepatitis B Core, Hepatitis B Surface Antigen, Cytomegalovirus B, HIV-1 gag, HIV-1 nef, HIV-1 env, HIV-1 gp41-1, HIV-1 p24, HIV-1 MN gp120, HIV-2 env, HIV-2 gp 36, HCV Core, HCV NS4, HCV NS3, HCV p22 nucleocapsid, HPV L1 capsid, HSV-1 gD, HSV-1 gG, HSV-2 gG, HSV-II, Influenza A (HIN1), Influenza A (H3N2), Influenza B, Parainfluenza Virus Type 1, Epstein Barr virus capsid antigen, Epstein Barr virus, Poxviridae Variola major, Poxviridae Variola minor, Rotavirus, Rubella virus, Respiratory Syncytial Virus, Surface Antigens of the Syphilis spirochete, Mumps Virus Antigen, Varicella zoster Virus Antigen and Filoviridae.

Other parasitic pathogens such as Chlamydia trachomatis, Plasmodium falciparum, and Toxoplasma gondii may also provide antigens for the methodologies contemplated by the present invention. Numerous bacterial and viral, and other microbe-generated antigens are available from commercial suppliers such as Research Diagnostics, Inc. (Flanders, N.J.).

Toxins, toxoids, or antigenic portions of either, utilized within the methodologies of the present invention include those produced by bacteria, such as diphteria toxin, tetanus toxin, botulin toxin and enterotoxin B; those produced by plants, such as Ricin toxin from the castor bean Ricinus cummunis. Mycotoxins, produced by fungi, that may serve in the present invention include diacetoxyscirpenol (DAS), Nivalenol, 4-Deoxynivalenol (DON), and T-2 Toxin. Other toxins and toxoids produced by or derived from other plants, snakes, fish, frogs, spiders, scorpions, blue-green algae, snails may also be incorporated into the methodologies of the present invention.

Additionally, epitopes identified by the methodologies of the present invention can be those derived from antigens that serve as markers for particular cell types, or as targets for an agent interacting with that cell type. Examples include Human Leukocyte Antigens (HLA markers), MHC Class I and Class II, the numerous CD markers useful for identifying T-cells and the physiological states thereof. Alternatively, antigens involved with the methodologies of the present invention can be those antibodies that serve as “markers” for a particular disease or condition, or as targets of a therapeutic agent. Examples include Prostate Specific Antigen, Pregnancy specific beta 1 glycoprotein (SP1), Thyroid Microsomal Antigen, and Urine Protein 1. Antigens may include those defined as “self” implicated in autoimmune diseases. Haptens, low molecular weight compounds such as drugs or antibiotics that are too small to cause an immune response unless they are coupled with much larger entities, can be involved in the methodologies contemplated by the present invention. See ROITT ET AL., IMMUNOLOGY (5th ed., 1998); BENJAMINI ET AL., IMMUNOLOGY, A SHORT COURSE (3rd ed., 1996).

Also within the scope of the present invention are databases comprising any portion or all of the data obtained by the methodologies of the present invention. A particular embodiment of the involves methods of doing business by providing a customer with any portion or all of the data obtained by the methodologies of the present invention. Another embodiment of the present invention involves any computer system and any form of recordable medium comprising any portion or all of the data obtained by the methodologies of the present invention. The term “recordable medium” includes, but is not limited to, any book, floppy disk, CD-ROM, ZIP disk, and any functional equivalent thereof.

### EXAMPLE

Without further elaboration, it is believed that one skilled in the art, using the preceding description, can utilize the present invention to the fullest extent. The following example is illustrative only, and not limiting of the remainder of the disclosure in any way whatsoever.

Human Papillomavirus (HPV) E2

The papillomaviruses are a family of small DNA viruses that cause genital warts and have been associated with cervical carcinomas. The E2 protein of HPV regulates viral transcription and is required for viral replication. Thus, molecules that block the binding of E2 to DNA can be useful therapeutic agents against HPV. The protein rather than the DNA should be chosen as a target, because it is expected that agents with greater selectivity would be found that bind to the protein rather than the DNA.

An ¹⁵N-labeled human papillomavirus E2 protein can be produced via recombinant technologies known to persons of ordinary skill in the art and overexpressed in bacteria using the T7 expression system. ¹⁵N-Alanine-labeled protein can be isolated from bacteria grown on a minimal medium. The protein can be purified from the bacterial cell lysate using an S-sepharose FastFlow column pre-equilibrated with buffer (50 mM Tris, 100 mM NaCl, 1 mM EDTA, pH=8.3).

The protein can be eluted with a linear gradient of 100-500 mM NaCl in buffer, pooled, and applied to a Mono-S column at a pH=7.0. The protein can be eluted with a salt gradient (100-500 mM), concentrated to 0.3 mM, and exchanged into a TRIS (50 mM, pH=7.0 buffered H₂O/D₂O (9/1) solution containing sodium azide (0.5%).

Characterization of the E2 Binding Domain using NMR Correlation Spectral Analysis

The E2 protein can be prepared in accordance with the procedures outlined above. The protein solutions used in the screening assay may contain ¹⁵N-Alanine-labeled E2 (0.3 mM), acetohydroxamic acid (500 mM), CaCl₂ (20 mM), and sodium azide (0.5%) in a H₂O/D₂O (9/1) TRIS buffered solution (50 mM, pH=7.0), and/or any other components known to persons of ordinary skill in the art.

Two-dimensional ¹⁵N NMR spectra can be generated at 29° C. on a Bruker AMX500 NMR spectrometer equipped with a triple resonance probe and Bruker sample changer. A delay of 1 second between scans and 8 scans per free induction decay (fid) can be employed in the data collection. All NMR spectra can be processed and analyzed on Silicon Graphics computers using software.

Spectra can be acquired for the ¹⁵N-Alanine-labeled E2 molecule in the absence of any ligand as described above. Similar experiments are run with recombinant E2 in which different amino acids are labeled.

The various labeled E2 proteins may then be exposed any member of a database of known ligands to E2. Stock solutions of the compounds can be made at 100 mM and 1M. One must note, however, that the pH of the 1M stock solution should be adjusted with acetic acid and ethanolamine so that no pH change will be observed upon a 1/10 dilution with a 100 mM phosphate buffered solution (pH=7.0). It is important to adjust the pH, because small changes in pH can alter the chemical shifts of the biomolecules and complicate the interpretation of the NMR data.

Additional spectra can be acquired for the ¹⁵N-labeled E2 target molecule in the presence of any one of-the known ligands as described above.

Comparisons of the spectra acquired for the ¹⁵N-labeled E2 protein in the absence of any ligand and the spectra acquired for the ¹⁵N-labeled E2 protein in the presence of any ligand may then be undertaken. In particular, comparisons between and among spectra should focus upon varying shifts in resonance suggesting what portions of the labeled E2 are involved in binding with a ligand. One may also compare the spectral data with the known sequence of the E2 protein. Via such comparisons, one may characterize the binding domain of E2.

What is claimed is:

1. A method for characterizing a binding domain of a labeled, recombinant protein, comprising comparing at least one nuclear magnetic resonance spectrum of a labeled, recombinant protein in the absence of a ligand with a nuclear magnetic resonance spectrum of a labeled, recombinant protein in the presence of a ligand.

2. A method according to claim 1, wherein the recombinant protein is a recombinant protein labeled at multiple sites or a recombinant protein labeled at alternate sites.

3. A method according to claim 1, wherein the recombinant protein is a tissue plasminogen activator receptor and the ligand is tissue plasminogen activator.

4. A method according to claim 1, wherein the recombinant protein is tumor necrosis factor and the ligand is any antibody that binds to tumor necrosis factor.

5. A method according to claim 1, wherein the recombinant protein is erythropoietin receptor and the ligand is erythropoietin or an erythropoietin mimetic.

6. A method according to claim 1, wherein the recombinant protein or the ligand is a diagnostic compound or a therapeutic compound.

7. A method according to claim 6, wherein the diagnostic or therapeutic compound is selected from at least one immunoglobulin, at least one fragment of an immunoglobulin, at least one integrin, at least one antigen, at least one growth factor, at least one cell cycle protein, at least one cytokine, at least one hormone, at last one neurotransmitter, or at least one receptor or at least one fusion proteins thereof, at least one blood protein, at least one antimicrobial, at least one chemokine, or any structural or functional analog thereof.

8. A method according to claim 7, wherein the immunoglobulin is at least one of IgG1, IgG2, IgG3, IgG4, IgA1, IgA2, slgA, IgD, IgE, or any structural or functional analog thereof.

9. A method according to claim 7, wherein the fragment of immunoglobulin is selected from at least one of F(ab′)₂, Fab′, Fab, Fc, Facb, pFc′, Fd, Fv, or any structural or functional analog thereof.

10. A method according to claim 7, wherein the integrin is selected from at least one of αv, β1, β2, β3, β4, β5, or any structural or functional analog thereof.

11. A method according to claim 7, wherein the antigen is selected from at least one of a cancer-specific antigen, a viral antigen, a bacterial antigen, a chemical antigen, or any structural or functional analog thereof.

12. A method according to claim 7, wherein the growth factor is selected from at least one of an insulin-like growth factor, a platelet-derived growth factor, a fibroblast growth factor 2, an epidermal growth factor, a tumor growth factor, a nerve growth factors, a vascular endothelial growth factor, or any structural or functional analog thereof.

13. A method according to claim 7, wherein the cell cycle protein is at least one selected from a p13, a p27, a p34, a p53, a p60, a p80, a histone H1, a centrosomal protein, a lamin, a CDK, a CDK inhibitor, a umor suppressor gene, or any structural or functional analog thereof.

14. A method according to claim 7, wherein the cytokine is at least one selected from an IL-1, an IL-2, an IL-3, an IL-4, an IL-5, an IL-6, an IL-7, an IL-8, an IL-9, an IL-10, an IL-11, an IL-12, an IL-13, an IL-14, an IL-15, an IL-16, an IL-17, an IL-18, an IL-19, an IL-20, an IL-21, an IL-22, an IL-23, an interferon, an interleukin, a lymphokine, a cell signal molecule, or any structural or functional analog thereof.

15. A method according to claim 7, wherein the hormone is selected from at least one a parathyroid hormone, a follicle stimulating hormone, an estrogen, a progesterone, a testosterone, an aldosterone, an antidiuretic hormone, a cortisols, a human chorionic gonadotropin, a parathormone, a growth hormone, a secretin, a thyroid hormone, or any structural or functional analog thereof.

16. A method according to claim 7, wherein the neurotransmitter is selected from at least one of a GABA, an enkephalins, or any structural or functional analog thereof.

17. A method according to claim 7, wherein the receptor or fusion protein thereof is selected from at least one of a chemokine receptor, a cytokine receptor, a cell cycle protein receptors, a growth factor receptor, a hormone receptor, a neurotransmitter receptor, a fusion protein to any one of the foregoing, or any structural or functional analog thereof.

18. A method according to claim 7, wherein the blood protein is at least one selected from a TPO, a tPA, or any structural or functional analog thereof.

19. A method according to claim 7, wherein the antimicrobial is at least one of an antibiotic, an antifungal, an antiprotozoal, an antiviral, or any structural or functional analog thereof.

20. A method according to claim 1, wherein the labeled, recombinant protein is an isotopically labeled protein.

21. A method according to claim 20,wherein the label is selected from at least one of ¹⁵N, ¹³C, ³II, ³³S, ¹⁷O, or ²II.

22. A method according to claim 1, wherein the comparing step is accomplished via a computer.

23. A method according to claim 22, wherein the the comparing step optionally further comprises includes the use of data obtained from X-ray crystallography analysis.

24. Recordable media, comprising data corresponding to at least one binding domain characterization obtained by a method according to claim 1.

25. A binding domain of a protein corresponding to at least a portion of the data according to claim 24.

26. A computer system, comprising recordable media according to claim 24.

27. A database comprising data corresponding to at least one binding domain characterization obtained by a method according to claim 1.

28. A business method, comprising the step of performing for a customer at least one method according to claim 1.
